SEQUENCE LISTING (101 sequences)

SEQ ID NO: 1 - protein, 5 aa, Artificial Sequence
  source: /note=“Description of Artificial Sequence: Synthetic peptide”
SYYWS

SEQ ID NO: 2 - protein, 16 aa, Artificial Sequence
  source: /note=“Description of Artificial Sequence: Synthetic peptide”
YIYYSGSTNYNPSLKS

SEQ ID NO: 3 - protein, 11 aa, Artificial Sequence
  source: /note=“Description of Artificial Sequence: Synthetic peptide”
SGYYTHDAFDI

SEQ ID NO: 4 - protein, 5 aa, Artificial Sequence
  source: /note=“Description of Artificial Sequence: Synthetic peptide”
GYYMH

SEQ ID NO: 5 - protein, 17 aa, Artificial Sequence
  source: /note=“Description of Artificial Sequence: Synthetic peptide”
WINPNSGGTNYAQKFQG

SEQ ID NO: 6 - protein, 12 aa, Artificial Sequence
  source: /note=“Description of Artificial Sequence: Synthetic peptide”
DCGSYYGDWFDP

SEQ ID NO: 7 - protein, 5 aa, Artificial Sequence
  source: /note=“Description of Artificial Sequence: Synthetic peptide”
TYWIG

SEQ ID NO: 8 - protein, 17 aa, Artificial Sequence
  source: /note=“Description of Artificial Sequence: Synthetic peptide”
IIYPGDSETRYSPSFQG

SEQ ID NO: 9 - protein, 10 aa, Artificial Sequence
  source: /note=“Description of Artificial Sequence: Synthetic peptide”
GSGISTPMDV

SEQ ID NO: 10 - protein, 5 aa, Artificial Sequence
  source: /note=“Description of Artificial Sequence: Synthetic peptide”
GYYMH

SEQ ID NO: 11 - protein, 17 aa, Artificial Sequence
  source: /note=“Description of Artificial Sequence: Synthetic peptide”
WINPNSGGTNYAQKFQG

SEQ ID NO: 12 - protein, 12 aa, Artificial Sequence
  source: /note=“Description of Artificial Sequence: Synthetic peptide”
DCGSYYGDWFDP

SEQ ID NO: 13 - protein, 5 aa, Artificial Sequence
  source: /note=“Description of Artificial Sequence: Synthetic peptide”
SYDMH

SEQ ID NO: 14 - protein, 16 aa, Artificial Sequence
  source: /note=“Description of Artificial Sequence: Synthetic peptide”
AIGTAGDTYYPGSVKG

SEQ ID NO: 15 - protein, 13 aa, Artificial Sequence
  source: /note=“Description of Artificial Sequence: Synthetic peptide”
GHYDSGGYGAFDI

SEQ ID NO: 16 - protein, 7 aa, Artificial Sequence
  source: /note=“Description of Artificial Sequence: Synthetic peptide”
NARMGVS

SEQ ID NO: 17 - protein, 16 aa, Artificial Sequence
  source: /note=“Description of Artificial Sequence: Synthetic peptide”
HIFSNDEKSYSTSLKS

SEQ ID NO: 18 - protein, 9 aa, Artificial Sequence
  source: /note=“Description of Artificial Sequence: Synthetic peptide”
IMRELPFDY

SEQ ID NO: 19 - protein, 5 aa, Artificial Sequence
  source: /note=“Description of Artificial Sequence: Synthetic peptide”
GYYMH

SEQ ID NO: 20 - protein, 17 aa, Artificial Sequence
  source: /note=“Description of Artificial Sequence: Synthetic peptide”
WINPNSGGTNYAQKFQG

SEQ ID NO: 21 - protein, 12 aa, Artificial Sequence
  source: /note=“Description of Artificial Sequence: Synthetic peptide”
DCGSYYGDWFDP

SEQ ID NO: 22 - protein, 5 aa, Artificial Sequence
  source: /note=“Description of Artificial Sequence: Synthetic peptide”
SYDMH

SEQ ID NO: 23 - protein, 16 aa, Artificial Sequence
  source: /note=“Description of Artificial Sequence: Synthetic peptide”
AIGTAGDTYYPGSVKG

SEQ ID NO: 24 - protein, 13 aa, Artificial Sequence
  source: /note=“Description of Artificial Sequence: Synthetic peptide”
GHYDSSGYGAFDI

SEQ ID NO: 25 - protein, 5 aa, Artificial Sequence
  source: /note=“Description of Artificial Sequence: Synthetic peptide”
SYDMH

SEQ ID NO: 26 - protein, 16 aa, Artificial Sequence
  source: /note=“Description of Artificial Sequence: Synthetic peptide”
AIGTAGDTYYPGSVKG

SEQ ID NO: 27 - protein, 13 aa, Artificial Sequence
  source: /note=“Description of Artificial Sequence: Synthetic peptide”
GHYDSGGYGAFDI

SEQ ID NO: 28 - protein, 11 aa, Artificial Sequence
  source: /note=“Description of Artificial Sequence: Synthetic peptide”
RASQSVSSYLA

SEQ ID NO: 29 - protein, 7 aa, Artificial Sequence
  source: /note=“Description of Artificial Sequence: Synthetic peptide”
DASNRAT

SEQ ID NO: 30 - protein, 9 aa, Artificial Sequence
  source: /note=“Description of Artificial Sequence: Synthetic peptide”
QQRSNWPLT

SEQ ID NO: 31 - protein, 12 aa, Artificial Sequence
  source: /note=“Description of Artificial Sequence: Synthetic peptide”
RASQSVSSSYLA

SEQ ID NO: 32 - protein, 7 aa, Artificial Sequence
  source: /note=“Description of Artificial Sequence: Synthetic peptide”
GASSRAT

SEQ ID NO: 33 - protein, 9 aa, Artificial Sequence
  source: /note=“Description of Artificial Sequence: Synthetic peptide”
QQYGSSPLT

SEQ ID NO: 34 - protein, 17 aa, Artificial Sequence
  source: /note=“Description of Artificial Sequence: Synthetic peptide”
KSSQSVLYSSINKNYLA

SEQ ID NO: 35 - protein, 7 aa, Artificial Sequence
  source: /note=“Description of Artificial Sequence: Synthetic peptide”
WASTRES

SEQ ID NO: 36 - protein, 9 aa, Artificial Sequence
  source: /note=“Description of Artificial Sequence: Synthetic peptide”
QQYYSTPYT

SEQ ID NO: 37 - protein, 12 aa, Artificial Sequence
  source: /note=“Description of Artificial Sequence: Synthetic peptide”
RASQSVSSSYLA

SEQ ID NO: 38 - protein, 7 aa, Artificial Sequence
  source: /note=“Description of Artificial Sequence: Synthetic peptide”
GASSRAT

SEQ ID NO: 39 - protein, 9 aa, Artificial Sequence
  source: /note=“Description of Artificial Sequence: Synthetic peptide”
QQYGSSPLT

SEQ ID NO: 40 - protein, 11 aa, Artificial Sequence
  source: /note=“Description of Artificial Sequence: Synthetic peptide”
RASQGISSYLA

SEQ ID NO: 41 - protein, 7 aa, Artificial Sequence
  source: /note=“Description of Artificial Sequence: Synthetic peptide”
TASTLQS

SEQ ID NO: 42 - protein, 10 aa, Artificial Sequence
  source: /note=“Description of Artificial Sequence: Synthetic peptide”
QQYYSYPPLT

SEQ ID NO: 43 - protein, 11 aa, Artificial Sequence
  source: /note=“Description of Artificial Sequence: Synthetic peptide”
RASQSISSWLA

SEQ ID NO: 44 - protein, 7 aa, Artificial Sequence
  source: /note=“Description of Artificial Sequence: Synthetic peptide”
KASSLES

SEQ ID NO: 45 - protein, 9 aa, Artificial Sequence
  source: /note=“Description of Artificial Sequence: Synthetic peptide”
QQYNSYSWT

SEQ ID NO: 46 - protein, 12 aa, Artificial Sequence
  source: /note=“Description of Artificial Sequence: Synthetic peptide”
RASQSVSSSYLA

SEQ ID NO: 47 - protein, 7 aa, Artificial Sequence
  source: /note=“Description of Artificial Sequence: Synthetic peptide”
GASSRAT

SEQ ID NO: 48 - protein, 9 aa, Artificial Sequence
  source: /note=“Description of Artificial Sequence: Synthetic peptide”
QQYGSSPLT

SEQ ID NO: 49 - protein, 11 aa, Artificial Sequence
  source: /note=“Description of Artificial Sequence: Synthetic peptide”
RASQGISSYLA

SEQ ID NO: 50 - protein, 7 aa, Artificial Sequence
  source: /note=“Description of Artificial Sequence: Synthetic peptide”
AASTLQS

SEQ ID NO: 51 - protein, 10 aa, Artificial Sequence
  source: /note=“Description of Artificial Sequence: Synthetic peptide”
QQYYSYPPLT

SEQ ID NO: 52 - protein, 11 aa, Artificial Sequence
  source: /note=“Description of Artificial Sequence: Synthetic peptide”
RASQGISSYLA

SEQ ID NO: 53 - protein, 7 aa, Artificial Sequence
  source: /note=“Description of Artificial Sequence: Synthetic peptide”
TASTLQS

SEQ ID NO: 54 - protein, 10 aa, Artificial Sequence
  source: /note=“Description of Artificial Sequence: Synthetic peptide”
QQYYSYPPLT

SEQ ID NO: 55 - protein, 119 aa, Artificial Sequence
  source: /note=“Description of Artificial Sequence: Synthetic polypeptide”
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGSGYYTHDAFDIWGQGTMVTVSS

SEQ ID NO: 56 - protein, 121 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDCGSYYGDWFDPWGQGTLVTVSS

SEQ ID NO: 57 - protein, 119 aa, Artificial Sequence
  source: /note=“Description of Artificial Sequence: Synthetic polypeptide”
EVQLVQSGAEVKKPGESLKISCKGSGYGFITYWIGWVRQMPRKGLEWMGIIYPGDSETRYSPSFQGQVTISADKSINTAYLQWSSLKASDTAIYYCAGGSGISTPMDVWGQGTTVTVSS

SEQ ID NO: 58 - protein, 121 aa, Artificial Sequence; identical to SEQ ID NO: 56
  source: /note=“Description of Artificial Sequence: Synthetic polypeptide”

SEQ ID NO: 59 - protein, 121 aa, Artificial Sequence
  source: /note=“Description of Artificial Sequence: Synthetic polypeptide”
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARGHYDSGGYGAFDIWGQGTMVTVSS

SEQ ID NO: 60 - protein, 39 aa, Artificial Sequence
  source: /note=“Description of Artificial Sequence: Synthetic polypeptide”
VLTMTNMDPVDTATYYCARIMRELPFDYWGQGTLVTVSS

SEQ ID NO: 61 - protein, 121 aa, Artificial Sequence
  source: /note=“Description of Artificial Sequence: Synthetic polypeptide”
QVQLVQSGAEVKKPGASVKVSCKASGYTFTGYYMHWVRQAPGQGLEWMGWINPNSGGTNYAQKFQGRVTMTRDTSISTVYMELSRLRSDDTAVYYCARDCGSYYGDWFDPWGQGTLVTVSS

SEQ ID NO: 62 - protein, 121 aa, Artificial Sequence
  source: /note=“Description of Artificial Sequence: Synthetic polypeptide”
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARGHYDSSGYGAFDIWGQGTMVTVSS

SEQ ID NO: 63 - protein, 121 aa, Artificial Sequence; identical to SEQ ID NO: 59
  source: /note=“Description of Artificial Sequence: Synthetic polypeptide”

SEQ ID NO: 64 - protein, 107 aa, Artificial Sequence
  source: /note=“Description of Artificial Sequence: Synthetic polypeptide”
EIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 65 - protein, 108 aa, Artificial Sequence
  source: /note=“Description of Artificial Sequence: Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 66 - protein, 113 aa, Artificial Sequence
  source: /note=“Description of Artificial Sequence: Synthetic polypeptide”
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTRLEIK

SEQ ID NO: 67 - protein, 107 aa, Artificial Sequence
  source: /note=“Description of Artificial Sequence: Synthetic polypeptide”
DIQMTQSPSSLSASVGDRVTITCQASQDISNYLNWYQQKPGKAPKLLIYDASNLETGVPSRFSGSGSGTDFTFTISSLQPEDIATYYCQQYDNLPFTFGQGTKVEIK

SEQ ID NO: 68 - protein, 108 aa, Artificial Sequence
  source: /note=“Description of Artificial Sequence: Synthetic polypeptide”
DIQLTQSPSFLSASVGDRVTITCRASQGISSYLAWYQQKPGKAPKLLIYTASTLQSGVPSRFSGSGSGTEFTLTISSLQPEDFATYYCQQYYSYPPLTFGPGTKVDIK

SEQ ID NO: 69 - protein, 107 aa, Artificial Sequence
  source: /note=“Description of Artificial Sequence: Synthetic polypeptide”
DIQMTQSPSTLSASVGDRVTITCRASQSISSWLAWYQQKPGKAPKLLIYKASSLESGVPSRFSGSGSGTEFTLTISSLQPDDFATYYCQQYNSYSWTFGQGTKVEIK

SEQ ID NO: 70 - protein, 108 aa, Artificial Sequence
  source: /note=“Description of Artificial Sequence: Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 71 - protein, 108 aa, Artificial Sequence
  source: /note=“Description of Artificial Sequence: Synthetic polypeptide”
AIRMTQSPSSFSASTGDRVTITCRASQGISSYLAWYQQKPGKAPKLLIYAASTLQSGVPSRFSGSGSGTDFTLTISCLQSEDFATYYCQQYYSYPPLTFGGGTKVEIK

SEQ ID NO: 72 - protein, 108 aa, Artificial Sequence
  source: /note=“Description of Artificial Sequence: Synthetic polypeptide”
DIQLTQSPSFLSASVGDRVTITCRASQGISSYLAWYQQKPGKAPKLLIYTASTLQSGVPSRFSGSGSGTEFTLTISCLQSEDFATYYCQQYYSYPPLTFGQGTKVEIK

SEQ ID NO: 73 - DNA, 357 bp, Artificial Sequence
  source: /note=“Description of Artificial Sequence: Synthetic polynucleotide”
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcggggagtggttattatacccatgatgcttttgatatctggggccaagggacaatggtcaccgtctcttct

SEQ ID NO: 74 - DNA, 363 bp, Artificial Sequence
  source: /note=“Description of Artificial Sequence: Synthetic polynucleotide”
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagattgtgggagctactacggggactggttcgacccctggggccagggaaccctggtcaccgtctcctct

SEQ ID NO: 75 - DNA, 357 bp, Artificial Sequence
  source: /note=“Description of Artificial Sequence: Synthetic polynucleotide”
gaggtgcagctggtgcagtctggagcagaggtgaaaaagccgggggagtctctgaagatctcctgtaagggttctggatacggctttatcacctactggatcggctgggtgcgccagatgcccaggaaaggcctggagtggatggggatcatctatcctggtgactctgaaaccagatacagcccgtccttccaaggccaggtcaccatctcagccgacaagtccatcaacaccgcctacctgcagtggagcagcctgaaggcctcggacaccgccatatattactgtgcggggggttcggggatttctacccctatggacgtctggggccaagggaccacggtcaccgtctcctct

SEQ ID NO: 76 - DNA, 363 bp, Artificial Sequence; identical to SEQ ID NO: 74
  source: /note=“Description of Artificial Sequence: Synthetic polynucleotide”

SEQ ID NO: 77 - DNA, 363 bp, Artificial Sequence
  source: /note=“Description of Artificial Sequence: Synthetic polynucleotide”
gaagtgcagctggtggagtctgggggaggcttggtacagcctggggggtccctgagactctcctgtgcagcctctggattcaccttcagtagctacgacatgcactgggtccgccaagctacaggaaaaggtctggagtgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagggggcactatgatagtggtggttatggggcttttgatatctggggccaagggacaatggtcaccgtctcttct

SEQ ID NO: 78 - DNA, 357 bp, Artificial Sequence
  source: /note=“Description of Artificial Sequence: Synthetic polynucleotide”
caggtcaccttgaaggagtctggtcctgtgctggtgaaacccacagagaccctcacgctgacctgcaccgtctctgggttctcactcagcaatgctagaatgggtgtgagctggatccgtcagcccccagggaaggccctggagtggcttgcacacattttttcgaatgacgaaaaatcctacagcacatctctgaagagcaggctcaccatctccaaggacacctccaaaagccaggtggtccttaccatgaccaacatggaccctgtggacacagccacatattactgtgcacggataatgagggagctaccctttgactactggggccagggaaccctggtcaccgtctcctct

SEQ ID NO: 79 - DNA, 363 bp, Artificial Sequence
  source: /note=“Description of Artificial Sequence: Synthetic polynucleotide”
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagtctacatggagctgagcaggctgagatctgacgacacggccgtgtattactgtgcgagagattgtgggagctactacggggactggttcgacccctggggccagggaaccctggtcaccgtctcctct

SEQ ID NO: 80 - DNA, 363 bp, Artificial Sequence
  source: /note=“Description of Artificial Sequence: Synthetic polynucleotide”
gaagtgcagctggtggagtctgggggaggcttggtacagcctggggggtccctgagactctcctgtgcagcctctggattcaccttcagtagctacgacatgcactgggtccgccaagctacaggaaaaggtctggagtgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagggggcactatgatagtagtggttatggggcttttgatatctggggccaagggacaatggtcaccgtctcttct

SEQ ID NO: 81 - DNA, 363 bp, Artificial Sequence; identical to SEQ ID NO: 77
  source: /note=“Description of Artificial Sequence: Synthetic polynucleotide”

SEQ ID NO: 82 - DNA, 319 bp, Artificial Sequence
  source: /note=“Description of Artificial Sequence: Synthetic polynucleotide”
gaaattgtaatgacacagtctccagccaccctgtctttgtctccaggggaaagagccaccctctcctgcagggccagtcagagtgttagcagctacttagcctggtaccaacagaaacctggccagctcccaggctcctcatctatgatgcatccaacagggccactggcatcccagccaggttcagtggcagtgggtctgggacagacttcactctcaccatcagcagcctagagcccgaagactttgcagtttattactgtcagcagcgtagcaactggcctctcactttcggccaagggaccaaggtggaaatcaa

SEQ ID NO: 83 - DNA, 324 bp, Artificial Sequence
  source: /note=“Description of Artificial Sequence: Synthetic polynucleotide”
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctcacttttggccaagggaccaaggtggaaatcaaa

SEQ ID NO: 84 - DNA, 339 bp, Artificial Sequence
  source: /note=“Description of Artificial Sequence: Synthetic polynucleotide”
gacatccagttgacccagtctccagactccctggctgtgtctctgggcgagagggccaccatcaactgcaagtccagccagagtgttttatacagctccatcaataagaactacttagcttggtaccagcagaaaccaggacagcctcctaagctgctcatttactgggcatctacccgggaatccggggtccctgaccgattcagtggcagcgggtctggcacagatttcactctcaccatcagcagcctgcaggctgaagatgtggcagtttattactgtcagcaatattatagtactccgtacactttcggccaagggacacgactggagattaaa

SEQ ID NO: 85 - DNA, 324 bp, Artificial Sequence
  source: /note=“Description of Artificial Sequence: Synthetic polynucleotide”
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacaaacttcactctcaccatcagcagactggagcctgaagattttgcagtgtattactgtcagcagtatggtagctcacctctcacgttcggccaagggaccaaggtggaaatcaaa

SEQ ID NO: 86 - DNA, 324 bp, Artificial Sequence
  source: /note=“Description of Artificial Sequence: Synthetic polynucleotide”
gacatccagctgacccagtctccatccttcctgtctgcatctgtaggagacagagtcaccatcacttgccgggccagtcagggcattagcagttatttagcctggtatcagcaaaaaccagggaaagcccctaagctcctgatctatactgcatccactttgcaaagtggggtcccatcaaggttcagcggcagtggatctgggacagaattcactctcacaatcagcagcctgcagcctgaagattttgcaacttattactgtcaacagtattatagttaccctccgctcactttcggccctgggaccaaagtggatatcaaa

SEQ ID NO: 87 - DNA, 321 bp, Artificial Sequence
  source: /note=“Description of Artificial Sequence: Synthetic polynucleotide”
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaa

SEQ ID NO: 88 - DNA, 324 bp, Artificial Sequence
  source: /note=“Description of Artificial Sequence: Synthetic polynucleotide”
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctcactttcggcggagggaccaaggtggagatcaaa

SEQ ID NO: 89 - DNA, 324 bp, Artificial Sequence
  source: /note=“Description of Artificial Sequence: Synthetic polynucleotide”
gccatccggatgacccagtctccatcctcattctctgcatctacaggagacagagtcaccatcacttgtcgggcgagtcagggtattagcagttatttagcctggtatcagcaaaaaccagggaaagcccctaagctcctgatctatgctgcatccactttgcaaagtggggtcccatcaaggttcagcggcagtggatctgggacagatttcactctcaccatcagctgcctgcagtctgaagattttgcaacttattactgtcaacagtattatagttaccctccgctcactttcggcggagggaccaaggtggagatcaaa

SEQ ID NO: 90 - DNA, 324 bp, Artificial Sequence
  source: /note=“Description of Artificial Sequence: Synthetic polynucleotide”
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatactgcatccactttgcaaagtggggtcccatcaaggttcagcggcagtggatctgggacagaattcactctcaccatcagctgcctgcagtctgaagattttgcaacttattactgtcaacagtattatagttaccctccgctcactttcggccaggggaccaaggtggagatcaaa

SEQ ID NO: 91-99 - skipped (9 entries)

SEQ ID NO: 100 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 101 - protein, 1286 aa, Artificial Sequence
  source: /note=“Description of Artificial Sequence: Synthetic polypeptide”
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGSGGSAWSHPQFE

(19) United States
(12) Patent Application Publication
(10) Pub. No.: US 2023/0279078 A1
(43) Pub. Date: Sep. 7, 2023

(54) SARS-COV-2 PROTEINS, ANTI-SARS-COV-2 ANTIBODIES, AND METHODS OF USING THE SAME

(71) Applicant: ASTRAZENECA UK LIMITED, CAMBRIDGE (GB)

(72) Inventors: GLEN HUGHES, CAMBRIDGE (GB); RUTH E. MARCH, CAMBRIDGE (GB); MARK ESSER, GAITHERSBURG, MD (US); PATRICK MCTAMNEY II, GAITHERSBURG, MD (US); YUEH-MING LOO, GAITHERSBURG, MD (US); REENA M. VARKEY, GAITHERSBURG, MD (US); QUN DU, GAITHERSBURG, MD (US); JAMES STEINHARDT, GAITHERSBURG, MD (US); SARAVANAN RAJAN, GAITHERSBURG, MD (US)

(21) Appl. No.: 18/006,054

(22) PCT Filed: Jul. 19, 2021
(86) PCT No.: PCT/EP2021/070176

§ 371 Date: Jan. 19, 2023

Related U.S. Application Data
(60) Provisional application No. 63/054,104, filed on Jul. 20, 2020.

(51) Int. Cl. C07K 16/10; C12N 15/63; G01N 33/569; G01N 33/543

(52) U.S. Cl. CPC C07K 16/10; C12N 15/63; G01N 33/56983; G01N 33/54388; C07K 2317/565; G01N 2469/20

(57) ABSTRACT

The present disclosure provides SARS-CoV-2 spike proteins and trimers containing such spike proteins. The present disclosure also provides antibodies and antigen-binding fragments thereof that specifically bind to the spike protein of SARS-CoV-2. The proteins, trimers, and antibodies can be used, for example, to detect anti-SARS-CoV-2 antibodies in sample obtained from a subject.

### 1. FIELD

The present disclosure relates to SARS-CoV-2 spike proteins, antibodies and antigen-binding fragments thereof that bind to SARS-CoV-2 spike protein, and methods of making and using the same, e.g., in the detection of anti-SARS-CoV-2 antibodies.

### 1.1 SEQUENCE LISTING

The instant application contains a Sequence Listing which has been submitted electronically in ASCII format and is hereby incorporated by reference in its entirety. Said ASCII copy, created on Jul. 9, 2021, is named COVID-101-WO-PCT_SL.txt and is 68,970 bytes in size.

### 2. BACKGROUND

A coronavirus 2019 (COVID19) pandemic caused by severe acute respiratory syndrome coronavirus 2 (SARS-CoV-2) has emerged. SARS-CoV-2 was first identified in Wuhan, China, in December 2019, and it quickly caused infections worldwide. The virus's mortality rate is currently uncertain, but the number of global cases and the deaths is staggering: as of July 2020, over fourteen million cases and six hundred thousand deaths have been confirmed globally. The virus is capable of person-to-person spread through small droplets from the nose or mouth, which are expelled when an infected person coughs, sneezes, or speaks. The incubation period (time from exposure to onset of symptoms) ranges from 0 to 24 days, with a mean of 3-5 days, but the virus may be contagious during this period. Symptoms include fever, coughing, and breathing difficulties. In some patients, the infection in the lung is severe causing severe respiratory distress or even death. However, many patients contracting the virus have only mild symptoms or are asymptomatic. Unfortunately, these patients may also be capable of spreading the virus to other people.

Currently, there is no approved vaccines and no specific treatment that has garnered widespread approval of the scientific and medical community, although several vaccine and antiviral approaches are being investigated. Thus, it is crucial to prevent the spread of SARS-CoV-2. It is hypothesized that patients who have been infected with SARS-CoV-2 and recovered may not be infective after recovery. Being able to identify people who will not become infective will be useful in preventing the spread of SARS-CoV-2. Some assays to test for anti-SARS-CoV-2 antibodies in patients are currently available, but they lack the desired sensitivity and specificity. Therefore, there is an urgent need for reagents and assays that are useful in identifying patients who have been infected with SARS-CoV-2.

### 3. SUMMARY

In some aspects, provided herein is an antibody or antigen-binding fragment thereof that specifically binds to the same epitope of the spike protein of SARS-CoV-2 as an antibody comprising a variable heavy chain (VH) and a variable light chain (VL) wherein the amino acid sequences of the VH and VL comprise the sequences of (a) SEQ ID NO: 55 and SEQ ID NO: 64, respectively; (b) SEQ ID NO: 56 and SEQ ID NO: 65, respectively; (c) SEQ ID NO: 57 and SEQ ID NO: 66, respectively; (d) SEQ ID NO: 58 and SEQ ID NO: 67, respectively; (e) SEQ ID NO: 59 and SEQ ID NO: 68, respectively; (f) SEQ ID NO: 60 and SEQ ID NO: 69, respectively; (g) SEQ ID NO: 61 and SEQ ID NO: 70, respectively; (h) SEQ ID NO:62 and SEQ ID NO: 71, respectively; or (i) SEQ ID NO: 63 and SEQ ID NO: 72, respectively.

In some aspects, provided herein is an antibody or antigen-binding fragment thereof that competitively inhibits binding of a reference antibody to the spike protein of SARS-CoV-2, wherein the reference antibody comprises a variable heavy chain (VH) and a variable light chain (VL), wherein the amino acid sequences of the VH and VL comprise the sequences of: (a) SEQ ID NO: 55 and SEQ ID NO: 64, respectively; (b) SEQ ID NO: 56 and SEQ ID NO: 65, respectively; (c) SEQ ID NO: 57 and SEQ ID NO: 66, respectively; (d) SEQ ID NO: 58 and SEQ ID NO: 67, respectively; (e) SEQ ID NO: 59 and SEQ ID NO: 68, respectively; (f) SEQ ID NO: 60 and SEQ ID NO: 69, respectively; (g) SEQ ID NO: 61 and SEQ ID NO: 70, respectively; (h) SEQ ID NO:62 and SEQ ID NO: 71, respectively; or (i) SEQ ID NO: 63 and SEQ ID NO: 72, respectively.

In some aspects, provided herein is an antibody or antigen-binding fragment thereof that specifically binds to the spike protein of SARS-CoV-2, comprising the VH-CDR1-3 and VL CDR1-3 amino acid sequences selected from the group consisting of: (a) SEQ ID NOs: 1, 2, and 3 and SEQ ID NOs: 28, 29, and 30, respectively; (b) SEQ ID NOs: 4, 5, and 6 and SEQ ID NOs: 31, 32, and 33, respectively; (c) SEQ ID NOs: 7, 8, and 9 and SEQ ID NOs: 34, 35, and 36, respectively; (d) SEQ ID NOs: 10, 11, and 12 and SEQ ID NOs: 37, 38, and 39, respectively; (e) SEQ ID NOs: 13, 14, and 15 and SEQ ID NOs: 40, 41, and 42, respectively; (f) SEQ ID NOs: 16, 17, and 18 and SEQ ID NOs: 43, 44, and 45, respectively; (g) SEQ ID NOs: 19, 20, and 21 and SEQ ID NOs: 46, 47, and 48, respectively; (h) SEQ ID NOs: 22, 23, and 24 and SEQ ID NOs: 49, 50, and 51, respectively; or (i) SEQ ID NOs: 25, 26, and 27 and SEQ ID NOs: 52, 53, and 54, respectively.

In some aspects, the antibody or antigen-binding fragment thereof provided herein comprises a variable heavy chain (VH) and a variable light chain (VL), wherein the variable heavy chain (VH) amino acid sequence is selected from the group consisting of: (a) SEQ ID NO: 55; (b) SEQ ID NO: 56; (c) SEQ ID NO: 57; (d) SEQ ID NO: 58; (e) SEQ ID NO: 59; (f) SEQ ID NO: 60; (g) SEQ ID NO: 61; (h) SEQ ID NO:62; and (i) SEQ ID NO: 63.

In some aspects, the antibody or antigen-binding fragment thereof provided herein comprises a variable heavy chain (VH) and a variable light chain (VL), wherein the variable light chain (VL) amino acid sequence selected from the group consisting of: (a) SEQ ID NO: 64; (b) SEQ ID NO: 65; (c) SEQ ID NO: 66; (d) SEQ ID NO: 67; (e) SEQ ID NO: 68; (f) SEQ ID NO: 69; (g) SEQ ID NO: 70; (h) SEQ ID NO: 71; and (i) SEQ ID NO: 72.

In some aspects, provided herein is an antibody or antigen-binding fragment thereof that specifically binds to the spike protein of SARS-CoV-2 comprising a variable heavy chain (VH) and a variable light chain (VL), wherein the variable heavy chain (VH) amino acid sequence is selected from the group consisting of: (a) SEQ ID NO: 55; (b) SEQ ID NO: 56; (c) SEQ ID NO: 57; (d) SEQ ID NO: 58; (e) SEQ ID NO: 59; (f) SEQ ID NO: 60; (g) SEQ ID NO: 61; (h) SEQ ID NO:62; and (i) SEQ ID NO: 63.

In some aspects, provided herein is an antibody or antigen-binding fragment thereof that specifically binds to the spike protein of SARS-CoV-2 comprising a variable heavy chain (VH) and a variable light chain (VL), wherein the variable light chain (VL) amino acid sequence selected from the group consisting of: (a) SEQ ID NO: 64; (b) SEQ ID NO: 65; (c) SEQ ID NO: 66; (d) SEQ ID NO: 67; (e) SEQ ID NO: 68; (f) SEQ ID NO: 69; (g) SEQ ID NO: 70; (h) SEQ ID NO: 71; and (i) SEQ ID NO: 72.

In some aspects, the antibody or antigen-binding fragment thereof provided herein comprises the variable heavy chain (VH) amino acid sequence and the variable light chain (VL) amino acid sequence selected from the group consisting of: (a) SEQ ID NO: 55 and SEQ ID NO: 64, respectively; (b) SEQ ID NO: 56 and SEQ ID NO: 65, respectively; (c) SEQ ID NO: 57 and SEQ ID NO: 66, respectively; (d) SEQ ID NO: 58 and SEQ ID NO: 67, respectively; (e) SEQ ID NO: 59 and SEQ ID NO: 68, respectively; (f) SEQ ID NO: 60 and SEQ ID NO: 69, respectively; (g) SEQ ID NO: 61 and SEQ ID NO: 70, respectively; (h) SEQ ID NO:62 and SEQ ID NO: 71, respectively; and (i) SEQ ID NO: 63 and SEQ ID NO: 72, respectively.

In some aspects, the antibody or antigen-binding fragment thereof provided herein cross-reacts with SARS-CoV.

In some aspects, the antibody or antigen-binding fragment thereof provided herein comprises a heavy chain constant region. In some aspects, the heavy chain constant region is selected from the group consisting of human immunoglobulins IgG and IgM.

In some aspects, the antibody or antigen-binding fragment thereof provided herein comprises a light chain constant region. In some aspects, the light chain constant region is a kappa light chain constant region. In some aspects, the light chain constant region is lambda light chain constant region.

In some aspects, the antibody or antigen-binding fragment thereof provided herein is a full length antibody.

In some aspects, the antibody or antigen-binding fragment thereof provided herein is an antigen-binding fragment. In some aspects, the antigen binding fragment comprises a Fab, Fab′, F(ab′)2, single chain Fv (scFv), disulfide linked Fv, V-NAR domain, IgNar, IgGΔCH2, minibody, F(ab′)3, tetrabody, triabody, diabody, single-domain antibody, (scFv)2, or scFv-Fc.

In some aspects, the antibody or antigen-binding fragment is isolated. In some aspects, the antibody or antigen-binding fragment is monoclonal. In some aspects, the antibody or antigen-binding fragment is recombinant.

In some aspects, the antibody or antigen-binding fragment provided herein does not neutralize SARS-CoV-2. In some aspects, the antibody or antigen-binding fragment provided herein does not neutralize a pseudovirus of SARS-CoV-2.

In some aspects, the antibody or antigen-binding fragment thereof provided herein further comprises a detectable label. In some aspects, said label is selected from the group consisting of enzyme label, gold particles, immunofluorescent label, chemiluminescent label, phosphorescent label, radiolabel, avidin/biotin, colored particles and magnetic particles. In some aspects, said label is detected by enzyme immunoassay, lateral flow test, radioimmunoassay, Western blot assay, immunofluorescent assay, immunoprecipitation assay, chemiluminescent assay, cytometry, or immunohistochemical assay.

In some aspects, provided herein is a polypeptide comprising the amino acid sequence of SEQ ID NO: 101. In some aspects, the polypeptide is isolated. In some aspects, the polypeptide is recombinantly produced or chemically synthesized. In some aspects, provided herein is a trimer comprising the polypeptide. In some aspects, the trimer is isolated. In some aspects, provided herein is an isolated polynucleotide comprising a nucleic acid molecule encoding the polypeptide.

In some aspects, provided herein is an isolated polynucleotide comprising a nucleic acid molecule encoding the heavy chain variable region and/or a nucleic acid molecule encoding the light chain variable region of an antibody or antigen-binding fragment thereof provided herein. In some aspects, provided herein is an isolated vector comprising a polynucleotide provided herein.

In some aspects, provided herein is a host cell comprising a polynucleotide provided herein, a vector provided herein, or a first vector comprising a nucleic acid molecule encoding the heavy chain variable region and a second vector comprising a nucleic acid molecule encoding the light chain variable region of an antibody or antigen-binding fragment thereof provided herein.

In some aspects, provided herein is a method of making an antibody or antigen-binding fragment thereof provided herein, or a protein provided herein comprising (a) culturing a cell provided herein and (b) isolating the antibody or antigen-binding fragment thereof or the protein from the cultured cell.

In some aspects, provided herein is a method for detecting an anti-SARS-CoV-2 antibody or antigen-binding fragment thereof in a sample comprising contacting the sample with a polypeptide or the trimer provided herein, optionally wherein the method further comprises detecting binding between the polypeptide or the trimer and the antibody or antigen-binding fragment thereof. In some aspects, the sample is a biological sample. In some aspects, the detecting method is an enzyme linked immunosorbent assay (ELISA). In some aspects, the detecting method is a lateral flow assay. In some aspects, the sample is from a human subject. In some aspects, the sample contains IgG antibodies or antigen-binding fragments thereof. In some aspects, the sample contains IgM antibodies or antigen-binding fragments thereof.

In some aspects, the method provided herein comprises the use of an antibody or antigen-binding fragment thereof provided herein as a control.

In some aspects, provided herein is a kit comprising (i) a SARS-CoV-2 spike protein provided herein, a trimer provided herein, or a polynucleotide provided herein and (ii) an antibody or antigen-binding fragment thereof that binds to SARS-CoV-2. In some aspects, the kit comprises an antibody or antigen-binding fragment thereof provided herein. In some aspects, the kit comprises an antibody or antigen-binding fragment thereof provided herein and a SARS-Co-V2 spike protein antigen. In some aspects, the kit comprises a notice that reflects approval for use or sale.

### 4. BRIEF DESCRIPTION OF THE FIGURES

FIG. 1 shows the dynamics of the positive rate of viral RNA and antibody of patients at different stages of disease.

FIG. 2 shows the binding of CV1-CV13 and R347 (control) antibodies to SARS2 trimer (left graph) and SARS receptor binding domain (RBD) (right graph).

FIG. 3 shows that CV1-CV12 antibodies do not neutralize pseudovirus. MEDI8897 is an anti-respiratory syncytial virus (RSV) antibody. Angiotensin converting enzyme-2 (ACE2) is the receptor for SARS-CoV-2.

FIG. 4 shows that the IgG and IgM formats of the CV7 antibody consistently produces positive results in an ELISA assay.

FIG. 5 shows the specificity of the ELISA for IgG antibodies that bind to the spike protein.

FIG. 6 shows steps that can be used in an assay to detect antibodies that bind to the spike protein of SARS-CoV-2.

### 5. DETAILED DESCRIPTION

Provided herein are SARS-CoV-2 spike proteins, antibodies (e.g., monoclonal antibodies) and antigen-binding fragments thereof that specifically bind to the spike protein, and methods of making, selecting, and using the same.

### 5.1 Terminology

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-SAR2-CoV-2 antibody,” “SARS-CoV-2 antibody” and “antibody that binds to SARS-CoV-2” are used interchangeably herein to refer to an antibody that is capable of binding to SARS-CoV-2. The extent of binding of a SARS-CoV-2 antibody to an unrelated, non-SARS-CoV-2 spike protein can be less than about 10% of the binding of the antibody to SARS-CoV-2 as measured, e.g., using ForteBio or Biacore. In some aspects provided herein, a SARS-CoV-2 antibody is also capable of binding to SARS-1. In some aspects provided herein, a SARS-CoV-2 antibody does not bind to SARS-1.

The terms “anti-spike protein of SAR2-CoV-2 antibody,” “SARS-CoV-2 spike protein antibody” and “antibody that binds to the spike protein of SARS-CoV-2” are used interchangeably herein to refer to an antibody that is capable of binding to the spike protein of SARS-CoV-2 with sufficient affinity such that the antibody is useful as a diagnostic agent in binding to the spike protein of SARS-CoV-2. The extent of binding of a SARS-CoV-2 spike protein antibody to an unrelated, non-SARS-CoV-2 spike protein can be less than about 10% of the binding of the antibody to SARS-CoV-2 spike protein as measured, e.g., using ForteBio or Biacore. In some aspects provided herein, a SARS-CoV-2 spike protein antibody is also capable of binding to the spike protein of SARS-1. In some aspects provided herein, a SARS-CoV-2 spike protein antibody does not bind to the spike protein of SARS-1.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The term “complementarity determining region” or “CDR” as used herein refers to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In some aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody/antigen-binding fragment thereof:antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

An antibody that “binds to the same epitope” as a reference antibody refers to an antibody that binds to the same amino acid residues as the reference antibody. The ability of an antibody to bind to the same epitope as a reference antibody can be determined by a hydrogen/deuterium exchange assay (see e.g., Coales et al. Rapid Commun. Mass Spectrom. 2009; 23: 639-647).

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails some complementarity between the antigen binding domain and the epitope. Accordingly, in some aspects, an antibody that “specifically binds” to the spike protein of SARS-CoV-2 can also bind to the spike protein of one or more related viruses (e.g., SARS-1) and/or can also bind to variants of the spike protein of SARS-CoV-2, but the extent of binding to an unrelated, non-SARS-CoV-2 spike protein is less than about 10% of the binding of the antibody to the spike protein of SARS-CoV-as measured, e.g., using ForteBio or Biacore.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition ELISA assays. An antibody can be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, amino acids with hydrophobic side chains include alanine (A), isoleucine (I), leucine (L), methionine (M), valine (V), phenylalanine (F), tryptophan (W), and tyrosine (Y). Amino acids with aliphatic hydrophobic side chains include alanine (A), isoleucine (I), leucine (L), methionine (M), and valine (V). Amino acids with aromatic hydrophobic side chains include phenylalanine (F), tryptophan (W), and tyrosine (Y).

As used herein, amino acids with polar neutral side chains include asparagine (N), cysteine (C), glutamine (Q), serine (S), and threonine (T).

As used herein, amino acids with electrically charged side chains include aspartic acid (D), glutamic acid (E), arginine (R), histidine (H), and lysine (K). Amino acids with acidic electrically charged side chains include aspartic acid (D) and glutamic acid (E). Amino acids with basic electrically charged side chains include arginine (R), histidine (H), and lysine (K).

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

Any compositions or methods provided herein can be combined with one or more of any of the other compositions and methods provided herein.

### 5.2 SARS-CoV-2 Spike Protein

The amino acid sequence a naturally occurring spike protein in SARS-CoV-2 is provided in SEQ ID NO: 100:

(SEQ ID NO: 100)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT.

Amino acids 1-12 of SEQ ID NO:100 are the signal peptide of the spike protein. Therefore, the mature version of the spike protein of SARS-CoV-2 contains amino acids 13-1273 of SEQ ID NO:100. Amino acids 13-1213 of SEQ ID NO:100 correspond to the extracellular domain; amino acids 1214-1234 correspond to the transmembrane domain; and amino acids 1235-1273 correspond to the cytoplasmic domain.

Provided herein are spike proteins with improved properties, e.g., improved expression (e.g., in cell culture) and improved efficacy in detecting anti-SARS-CoV-2 antibodies. In one aspect, a SARS-CoV-2 protein comprises the following sequence:

(SEQ ID NO: 101)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQGSGYIPEAPRDGQAYVRKDGEWVLLSTFLGRSLEVLFQGPGH
HHHHHHHSAWSHPQFEKGGGSGGGSGGSAWSHPQFE

In one aspect, a protein provided herein comprises the amino acid sequence set forth in SEQ ID NO:101. In one aspect, provided herein is a trimer comprising a protein comprising the amino acid sequence of SEQ ID NO:101.

### 5.3 Antibodies and Antigen-Binding Fragments Thereof

In a specific aspect, provided herein are antibodies (e.g., monoclonal antibodies, such as human antibodies) and antigen-binding fragments thereof that bind to the spike protein of SARS-CoV-2.

In some aspects, an antibody or antigen-binding fragment thereof is capable of binding to a spike protein comprising the amino acid sequence set forth in SEQ ID NO:100 and/or a spike protein comprising the amino acid sequence set forth in SEQ ID NO:101. In some aspects, an antibody or antigen-binding fragment thereof is capable of binding to a trimer comprising a spike protein comprising the amino acid sequence set forth in SEQ ID NO:100 and/or a spike protein comprising the amino acid sequence set forth in SEQ ID NO:101.

In some aspects, an antibody or antigen-binding fragment thereof described herein that binds to the spike protein of SARS-CoV-2 is capable of binding to the SARS2 trimer. In some aspects, an antibody or antigen-binding fragment thereof that binds to the spike protein of SARS-CoV-2 is capable of binding to the receptor binding domain (RBD, amino acids 334-526 of SEQ ID NO: 100) of the spike protein. In some aspects, an antibody or antigen-binding fragment thereof described herein that binds to the spike protein of SARS-CoV-2 is capable of binding to the SARS2 trimer and to the receptor binding domain (RBD) of the spike protein.

In some aspects, and antibody or antigen-binding fragment thereof described herein is capable of binding to a spike protein comprising the amino acid sequence set forth in SEQ ID NO: 101. In some aspects, an antibody or antigen-binding fragment thereof described herein is capable of binding to a SARS2 trimer comprising the amino acid sequence set forth in SEQ ID NO:101 (e.g., comprising 3 proteins each with amino acid sequence set forth in SEQ ID NO:101.)

In some aspects, an antibody or antigen-binding fragment thereof described herein binds to the spike protein of SARS-CoV-2 and comprises the six CDRs of an antibody listed in Tables 1 and 2 (i.e., the three VH CDRs of the antibody and the three VL CDRs of the same antibody).

In some aspects, an antibody or antigen-binding fragment thereof described herein binds to the spike protein of SARS-CoV-2, and comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 85% identical to the VH sequence of the same antibody in Table 3 and a VL comprising a sequence at least 85% identical to the VL sequence of the same antibody in Table 4.

In some aspects, an antibody or antigen-binding fragment thereof described herein binds to the spike protein of SARS-CoV-2, and comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 90% identical to the VH sequence of the same antibody in Table 3 and a VL comprising a sequence at least 90% identical to the VL sequence of the same antibody in Table 4. In some aspects, an antibody or antigen-binding fragment thereof described herein that binds to the spike protein of SARS-CoV-2, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 95% identical to the VH sequence of the same antibody in Table 3 and a VL comprising a sequence at least 95% identical to the VL sequence of the same antibody in Table 4.

In some aspects, an antibody or antigen-binding fragment thereof described herein binds to the spike protein of SARS-CoV-2, and comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 96% identical to the VH sequence of the same antibody in Table 3 and a VL comprising a sequence at least 96% identical to the VL sequence of the same antibody in Table 4. In some aspects, an antibody or antigen-binding fragment thereof described herein binds to the spike protein of SARS-CoV-2, and comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 97% identical to the VH sequence of the same antibody in Table 3 and a VL comprising a sequence at least 97% identical to the VL sequence of the same antibody in Table 4. In some aspects, an antibody or antigen-binding fragment thereof described herein binds to the spike protein of SARS-CoV-2, and comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 98% identical to the VH sequence of the same antibody in Table 3 and a VL comprising a sequence at least 98% identical to the VL sequence of the same antibody in Table 4. In some aspects, an antibody or antigen-binding fragment thereof described herein binds to the spike protein of SARS-CoV-2, comprises the six CDRs of an antibody listed in Tables 1 and 2 (i.e., the three VH CDRs of the antibody listed in Table 1 and the three VL CDRs of the same antibody listed in Table 2), and comprises a VH comprising a sequence at least 99% identical to the VH sequence of the same antibody in Table 3 and a VL comprising a sequence at least 99% identical to the VL sequence of the same antibody in Table 4.

TABLE 1
Variable heavy chain CDR amino acid sequences

| Antibody | VH-CDR1 | VH-CDR2 | VH-CDR3 |
|---|---|---|---|
| Clone 1 | SYYWS | YIYYSGSTNYNPSLKS | SGYYTHDAFDI |
| | (SEQ ID NO: 1) | (SEQ ID NO: 2) | (SEQ ID NO: 3) |
| Clone 2 | GYYMH | WINPNSGGTNYAQKFQG | DCGSYYGDWFDP |
| | (SEQ ID NO: 4) | (SEQ ID NO: 5) | (SEQ ID NO: 6) |
| Clone 4 | TYWIG | IIYPGDSETRYSPSFQG | GSGISTPMDV |
| | (SEQ ID NO: 7) | (SEQ ID NO: 8) | (SEQ ID NO: 9) |
| Clone 6 | GYYMH | WINPNSGGTNYAQKFQG | DCGSYYGDWFDP |
| | (SEQ ID NO: 10) | (SEQ ID NO: 11) | (SEQ ID NO: 12) |
| Clone 7 | SYDMH | AIGTAGDTYYPGSVKG | GHYDSGGYGAFDI |
| | (SEQ ID NO: 13) | SEQ ID NO: 14) | (SEQ ID NO: 15) |
| Clone 9 | NARMGVS | HIFSNDEKSYSTSLKS | IMRELPFDY |
| | (SEQ ID NO: 16) | (SEQ ID NO: 17) | (SEQ ID NO: 18) |
| Clone 10 | GYYMH | WINPNSGGTNYAQKFQG | DCGSYYGDWFDP |
| | (SEQ ID NO: 19) | (SEQ ID NO: 20) | (SEQ ID NO: 21) |
| Clone 11 | SYDMH | AIGTAGDTYYPGSVKG | GHYDSSGYGAFDI |
| | (SEQ ID NO: 22) | (SEQ ID NO: 23) | (SEQ ID NO: 24) |
| Clone 12 | SYDMH | AIGTAGDTYYPGSVKG | GHYDSGGYGAFDI |
| | (SEQ ID NO: 25) | (SEQ ID NO: 26) | (SEQ ID NO: 27) |

TABLE 2
Variable light chain CDR amino acid sequences

| Antibody | VH-CDR1 | VH-CDR2 | VH-CDR3 |
|---|---|---|---|
| Clone 1 | RASQSVSSYLA | DASNRAT | QQRSNWPLT |
| | (SEQ ID NO: 28) | (SEQ ID NO: 29) | (SEQ ID NO: 30) |
| Clone 2 | RASQSVSSSYLA | GASSRAT | QQYGSSPLT |
| | (SEQ ID NO: 31) | (SEQ ID NO: 32) | (SEQ ID NO: 33) |
| Clone 4 | KSSQSVLYSSINKNYLA | WASTRES | QQYYSTPYT |
| | (SEQ ID NO: 34) | (SEQ ID NO: 35) | (SEQ ID NO: 36) |
| Clone 6 | RASQSVSSSYLA | GASSRAT | QQYGSSPLT |
| | (SEQ ID NO: 37) | (SEQ ID NO: 38) | (SEQ ID NO: 39) |
| Clone 7 | RASQGISSYLA | TASTLQS | QQYYSYPPLT |
| | (SEQ ID NO: 40) | (SEQ ID NO: 41) | (SEQ ID NO: 42) |
| Clone 9 | RASQSISSWLA | KASSLES | QQYNSYSWT |
| | (SEQ ID NO: 43) | (SEQ ID NO: 44) | (SEQ ID NO: 45) |
| Clone 10 | RASQSVSSSYLA | GASSRAT | QQYGSSPLT |
| | (SEQ ID NO: 46) | (SEQ ID NO: 47) | (SEQ ID NO: 48) |
| Clone 11 | RASQGISSYLA | AASTLQS | QQYYSYPPLT |
| | (SEQ ID NO: 49) | (SEQ ID NO: 50) | (SEQ ID NO: 51) |
| Clone 12 | RASQGISSYLA | TASTLQS | QQYYSYPPLT |
| | (SEQ ID NO: 52) | (SEQ ID NO: 53) | (SEQ ID NO: 54) |

In some aspects, an antibody or antigen-binding fragment thereof described herein specifically binds to the same epitope of the spike protein of SARS-CoV-2 as an antibody comprising a variable heavy chain (VH) and/or a variable light chain (VL) of an antibody listed in Tables 3 and 4.

In some aspects, an antibody or antigen-binding fragment thereof described herein competitively inhibits binding of a reference antibody to the spike protein of SARS-CoV-2 antibody, wherein the reference antibody comprises a variable heavy chain (VH) listed in Table 3 and/or a variable light chain (VL), of an antibody listed in Tables 3 and 4.

TABLE 3
Variable heavy chain amino acid sequences

| Antibody | VH Amino Acid Sequence (SEQ ID NO) |
|---|---|
| Clone 1 | QVQLQESGPGLVKPSETLSLTCTVSGGSISSYYWSWIRQPPGKGLEWIGYI |
| | YYSGSTNYNPSLKSRVTISVDTSKNQFSLKLSSVTAADTAVYYCAGSGYY |
| | THDAFDIWGQGTMVTVSS |
| | (SEQ ID NO: 55) |
| Clone 2 | QVQLVQSGAEVKKPGASVKVSCKASGYTFTGYYMHWVRQAPGQGLEW |
| | MGWINPNSGGTNYAQKFQGRVTMTRDTSISTAYMELSRLRSDDTAVYY |
| | CARDCGSYYGDWFDPWGQGTLVTVSS |
| | (SEQ ID NO: 56) |
| Clone 4 | EVQLVQSGAEVKKPGESLKISCKGSGYGFITYWIGWVRQMPRKGLEWM |
| | GIIYPGDSETRYSPSFQGQVTISADKSINTAYLQWSSLKASDTAIYYCAGG |
| | SGISTPMDVWGQGTTVTVSS |
| | (SEQ ID NO: 57) |
| Clone 6 | QVQLVQSGAEVKKPGASVKVSCKASGYTFTGYYMHWVRQAPGQGLEW |
| | MGWINPNSGGTNYAQKFQGRVTMTRDTSISTAYMELSRLRSDDTAVYY |
| | CARDCGSYYGDWFDPWGQGTLVTVSS |
| | (SEQ ID NO: 58) |
| Clone 7 | EVQLVESGGGLVQPGGSLRLSCAASGFTFSSYDMHWVRQATGKGLEWV |
| | SAIGTAGDTYYPGSVKGRFTISRENAKNSLYLQMNSLRAGDTAVYYCAR |
| | GHYDSGGYGAFDIWGQGTMVTVSS |
| | (SEQ ID NO: 59) |
| Clone 9 | VLTMTNMDPVDTATYYCARIMRELPFDYWGQGTLVTVSS |
| | (SEQ ID NO: 60) |
| Clone 10 | QVQLVQSGAEVKKPGASVKVSCKASGYTFTGYYMHWVRQAPGQGLEW |
| | MGWINPNSGGTNYAQKFQGRVTMTRDTSISTVYMELSRLRSDDTAVYY |
| | CARDCGSYYGDWFDPWGQGTLVTVSS |
| | (SEQ ID NO: 61) |
| Clone 11 | EVQLVESGGGLVQPGGSLRLSCAASGFTFSSYDMHWVRQATGKGLEWV |
| | SAIGTAGDTYYPGSVKGRFTISRENAKNSLYLQMNSLRAGDTAVYYCAR |
| | GHYDSSGYGAFDIWGQGTMVTVSS |
| | (SEQ ID NO: 62) |
| Clone 12 | EVQLVESGGGLVQPGGSLRLSCAASGFTFSSYDMHWVRQATGKGLEWV |
| | SAIGTAGDTYYPGSVKGRFTISRENAKNSLYLQMNSLRAGDTAVYYCAR |
| | GHYDSGGYGAFDIWGQGTMVTVSS |
| | (SEQ ID NO: 63) |

TABLE 4
Variable light chain amino acid sequences

| Antibody | VLAmino Acid Sequence (SEQ ID NO) |
|---|---|
| Clone 1 | EIVMTQSPATLSLSPGERATLSCRASQSVSSYLAWYQQKPGQAPRLLIYD |
| | ASNRATGIPARFSGSGSGTDFTLTISSLEPEDFAVYYCQQRSNWPLTFGQG |
| | TKVEIK |
| | (SEQ ID NO: 64) |
| Clone 2 | EIVLTQSPGTLSLSPGERATLSCRASQSVSSSYLAWYQQKPGQAPRLLIYG |
| | ASSRATGIPDRFSGSGSGTDFTLTISRLEPEDFAVYYCQQYGSSPLTFGQG |
| | TKVEIK |
| | (SEQ ID NO: 65) |
| Clone 4 | DIQLTQSPDSLAVSLGERATINCKSSQSVLYSSINKNYLAWYQQKPGQPP |
| | KLLIYWASTRESGVPDRFSGSGSGTDFTLTISSLQAEDVAVYYCQQYYST |
| | PYTFGQGTRLEIK |
| | (SEQ ID NO: 66) |
| Clone 6 | DIQMTQSPSSLSASVGDRVTITCQASQDISNYLNWYQQKPGKAPKLLIYD |
| | ASNLETGVPSRFSGSGSGTDFTFTISSLQPEDIATYYCQQYDNLPFTFGQG |
| | TKVEIK |
| | (SEQ ID NO: 67) |
| Clone 7 | DIQLTQSPSFLSASVGDRVTITCRASQGISSYLAWYQQKPGKAPKLLIYTA |
| | STLQSGVPSRFSGSGSGTEFTLTISSLQPEDFATYYCQQYYSYPPLTFGPGT |
| | KVDIK |
| | (SEQ ID NO: 68) |
| Clone 9 | DIQMTQSPSTLSASVGDRVTITCRASQSISSWLAWYQQKPGKAPKLLIYK |
| | ASSLESGVPSRFSGSGSGTEFTLTISSLQPDDFATYYCQQYNSYSWTFGQG |
| | TKVEIK |
| | (SEQ ID NO: 69) |
| Clone 10 | EIVLTQSPGTLSLSPGERATLSCRASQSVSSSYLAWYQQKPGQAPRLLIYG |
| | ASSRATGIPDRFSGSGSGTDFTLTISRLEPEDFAVYYCQQYGSSPLTFGGG |
| | TKVEIK |
| | (SEQ ID NO: 70) |
| Clone 11 | AIRMTQSPSSFSASTGDRVTITCRASQGISSYLAWYQQKPGKAPKLLIYAA |
| | STLQSGVPSRFSGSGSGTDFTLTISCLQSEDFATYYCQQYYSYPPLTFGGG |
| | TKVEIK |
| | (SEQ ID NO: 71) |
| Clone 12 | DIQLTQSPSFLSASVGDRVTITCRASQGISSYLAWYQQKPGKAPKLLIYTA |
| | STLQSGVPSRFSGSGSGTEFTLTISCLOSEDFATYYCQQYYSYPPLTFGQG |
| | TKVEIK |
| | (SEQ ID NO: 72) |

In some aspects, an antibody or antigen-binding fragment thereof described herein binds to the spike protein of SARS-CoV-2 and comprises the VH of an antibody listed in Table 3. In some aspects, an antibody or antigen-binding fragment thereof described herein binds to the spike protein of SARS-CoV-2 and comprises the VL of an antibody listed in Table 4.

In some aspects, an antibody or antigen-binding fragment thereof described herein binds to the spike protein of SARS-CoV-2 and comprises the VH and the VL of an antibody listed in Tables 3 and 4 (i.e., the VH of the antibody and the VL of the same antibody).

In some aspects, an antibody or antigen-binding fragment thereof described herein may be described by its VL domain alone, or its VH domain alone, or by its 3 VL CDRs alone, or its 3 VH CDRs alone. See, for example, Rader C et al., (1998) PNAS 95: 8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352: 624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45: 572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In some aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, provided herein are antibodies and antigen-binding fragments thereof that specifically bind to the spike protein of SARS-CoV-2 and comprise the Chothia VH and VL CDRs of an antibody comprising the VH and VL sequences listed in Tables 3 and 4 (i.e., the VH of the antibody and the VL of the same antibody). In some aspects, antibodies or antigen-binding fragments thereof that specifically bind to the spike protein of SARS-CoV-2 comprise one or more CDRs, in which the Chothia and Kabat CDRs have the same amino acid sequence. In some aspects, provided herein are antibodies and antigen-binding fragments thereof that specifically bind to the spike protein of SARS-CoV-2 and comprise combinations of Kabat CDRs and Chothia CDRs.

In some aspects, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects, provided herein are antibodies and antigen-binding fragments thereof that specifically bind to the spike protein of SARS-CoV-2 and comprise the IMGT VH and VL CDRs of an antibody comprising the VH and VL sequences listed in Tables 3 and 4 (i.e., the VH of the antibody and the VL of the same antibody), for example, as described in Lefranc M-P (1999) supra and Lefranc M-P et al., (1999) supra).

In some aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, provided herein are antibodies or antigen-binding fragments thereof that specifically bind to the spike protein of SARS-CoV-2 and comprise VH and VL CDRs of an antibody comprising the VH and VL sequences listed in Tables 4 and 4 (i.e., the VH of the antibody and the VL of the same antibody) as determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, provided herein are antibodies or antigen-binding fragments thereof that specifically bind to the spike protein of SARS-CoV-2 and comprise VH and VL CDRs of an antibody comprising the VH and the VL sequences listed in Tables 3 and 4 (i.e., the VH of the antibody and the VL of the same antibody) as determined by the AbM numbering scheme.

In some aspects, provided herein are antibodies that comprise a heavy chain and/or a light chain. Non-limiting examples of human constant region sequences have been described in the art, e.g., see U.S. Pat. No. 5,693,780 and Kabat E A et al., (1991) supra.

With respect to the heavy chain, in some aspects, the heavy chain of an antibody described herein can be an alpha (α), delta (δ), epsilon (ε), gamma (γ) or mu (μ) heavy chain. In some aspects, the heavy chain of an antibody described can comprise a human alpha (α), delta (δ), epsilon (ε), gamma (γ) or mu (μ) heavy chain. In some aspects, an antibody described herein, which immunospecifically binds to the spike protein of SARS-CoV-2, comprises a heavy chain wherein the amino acid sequence of the VH domain comprises an amino acid sequence set forth in Table 3 and wherein the constant region of the heavy chain comprises the amino acid sequence of a human gamma (γ) heavy chain constant region (e.g., a human IgG1 heavy chain constant region). In some aspects, an antibody described herein, which immunospecifically binds to the spike protein of SARS-CoV-2, comprises a heavy chain wherein the amino acid sequence of the VH domain comprises an amino acid sequence set forth in Table 3 and wherein the constant region of the heavy chain comprises the amino acid sequence of a human IgM heavy chain constant region. In some aspects, an antibody described herein, which specifically binds to the spike protein of SARS-CoV-2, comprises a heavy chain wherein the amino acid sequence of the VH domain comprises a sequence set forth in Table 3, and wherein the constant region of the heavy chain comprises the amino acid of a human heavy chain described herein or known in the art.

In some aspects, the light chain of an antibody or antigen-binding fragment thereof described herein is a human kappa light chain or a human lambda light chain. In some aspects, an antibody described herein, which immunospecifically binds to the spike protein of SARS-CoV-2 comprises a light chain wherein the amino acid sequence of the VL domain comprises a sequence set forth in Table 4 and wherein the constant region of the light chain comprises the amino acid sequence of a human kappa or lambda light chain constant region.

In some aspects, an antibody or antigen-binding fragment thereof described herein, which immunospecifically binds to the spike protein of SARS-CoV-2 comprises a light chain wherein the amino acid sequence of the VL domain comprises a sequence set forth in Table 4, and wherein the constant region of the light chain comprises the amino acid sequence of a human kappa light chain constant region.

In some aspects, the light chain of an antibody described herein is a lambda light chain. In some aspects, an antibody described herein, which immunospecifically binds to the spike protein of SARS-CoV-2 comprises a light chain wherein the amino acid sequence of the VL domain comprises a sequence set forth in Table 4 and wherein the constant region of the light chain comprises the amino acid sequence of a human lambda light chain constant region.

In some aspects, an antibody described herein, which immunospecifically binds to the spike protein of SARS-CoV-2 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, an antibody described herein, which immunospecifically binds to the spike protein of SARS-CoV-2 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

In some aspects, an antibody or antigen-binding fragment thereof described herein, that specifically binds to the spike protein of SARS-CoV-2 does not neutralize SARS-CoV-2. In some aspects, an antibody or antigen-binding fragment thereof described herein, that specifically binds to the spike protein of SARS-CoV-2 does not neutralize a pseudovirus of SARS-CoV-2.

Competition binding assays can be used to determine whether two antibodies bind to overlapping epitopes. Competitive binding can be determined in an assay in which the immunoglobulin under test inhibits specific binding of a reference antibody to a common antigen, such as the spike protein of SARS-CoV-2 or SARS-CoV-2. Numerous types of competitive binding assays are known, for example: solid phase direct or indirect radioimmunoassay (RIA), solid phase direct or indirect enzyme immunoassay (EIA), sandwich competition assay (see Stahli C et al., (1983) Methods Enzymol 9: 242-253); solid phase direct biotin-avidin EIA (see Kirkland T N et al., (1986) J Immunol 137: 3614-9); solid phase direct labeled assay, solid phase direct labeled sandwich assay (see Harlow E & Lane D, (1988) Antibodies: A Laboratory Manual, Cold Spring Harbor Press); solid phase direct label RIA using I-125 label (see Morel G A et al., (1988) Mol Immunol 25(1): 7-15); solid phase direct biotin-avidin EIA (Cheung R C et al., (1990) Virology 176: 546-52); and direct labeled RIA. (Moldenhauer G et al., (1990) Scand J Immunol 32: 77-82). Typically, such an assay involves the use of purified antigen bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70%, 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener C et al., (1983) J Immunol 130: 2308-2315; Wagener C et al., (1984) J Immunol Methods 68: 269-274; Kuroki M et al., (1990) Cancer Res 50: 4872-4879; Kuroki M et al., (1992) Immunol Invest 21: 523-538; Kuroki M et al., (1992) Hybridoma 11: 391-407 and Antibodies: A Laboratory Manual, Ed Harlow E & Lane D editors supra, pp. 386-389.

In some aspects, a competition assay is performed using surface plasmon resonance (BIAcore®), e.g., by an ‘in tandem approach’ such as that described by Abdiche Y N et al., (2009) Analytical Biochem 386: 172-180, whereby antigen is immobilized on the chip surface, for example, a CMS sensor chip and the antibodies or antigen-binding fragments are then run over the chip. To determine if an antibody or antigen-binding fragment thereof competes with an antibody that binds to the spike protein of SARS-CoV-2 as described herein, the antibody or antigen-binding fragment is first run over the chip surface to achieve saturation and then the potential, competing antibody is added. Binding of the competing antibody or antigen-binding fragment thereof can then be determined and quantified relative to a non-competing control.

In another aspect, provided herein are antibodies that competitively inhibit (e.g., in a dose dependent manner) an antibody or antigen-binding fragment thereof described from binding to the spike protein of SARS-CoV-2 or to SARS-CoV-2, as determined using assays known to one of skill in the art or described herein (e.g., ELISA competitive assays, or suspension array or surface plasmon resonance assay).

In some aspects, an antigen-binding fragment as described herein that specifically binds to the spike protein of SARS-CoV-2, is selected from the group consisting of a Fab, Fab′, F(ab′)₂, and scFv, wherein the Fab, Fab′, F(ab′)₂, or scFv comprises a heavy chain variable region sequence and a light chain variable region sequence of an antibody or antigen-binding fragment thereof described herein that specifically binds to the spike protein of SARS-CoV-2 or to SARS-CoV-2. A Fab, Fab′, F(ab′)₂, or scFv can be produced by any technique known to those of skill in the art, including, but not limited to, those discussed in herein.

An antibody or antigen-binding fragment thereof that binds to the spike protein of SARS-CoV-2 can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I) carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labeled antibodies or antigen-binding fragments thereof can be used to detect the spike protein of SARS-CoV-2 or SARS-CoV-2 or antibodies that bind to SARS-CoV-2, as discussed elsewhere herein.

### 5.4 Protein and Antibody Production

Spike proteins and antibodies and antigen-binding fragments thereof that immunospecifically bind to the spike protein of SARS-CoV-2 can be produced by any method known in the art for the synthesis of proteins and antibodies and antigen-binding fragments thereof, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J et al., (2001) Molecular Cloning: A Laboratory Manual, Cold Spring Harbor Laboratory Press, Cold Spring Harbor, N.Y.; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some aspects, provided herein is a method of making a spike protein comprising the amino acid sequence set forth in SEQ ID NO:101, the method comprising culturing a cell or host cell described herein. In some aspects, provided herein is a method of making a spike protein comprising the amino acid sequence set forth in SEQ ID NO:101, the method comprising expressing (e.g., recombinantly expressing) the protein using a cell or host cell described herein (e.g., a cell or a host cell comprising polynucleotides encoding an antibody or antigen-binding fragment thereof described herein). In some aspects, the cell is an isolated cell. In some aspects, exogenous polynucleotides have been introduced into the cell. In some aspects, the method further comprises the step of separating or purifying the protein obtained from the cell, host cell, or culture.

In some aspects, provided herein is a method of making an antibody or antigen-binding fragment which immunospecifically binds to the spike protein of SARS-CoV-2 comprising culturing a cell or host cell described herein. In some aspects, provided herein is a method of making an antibody or antigen-binding fragment thereof which immunospecifically binds to the spike protein of SARS-CoV-2 comprising expressing (e.g., recombinantly expressing) the antibody or antigen-binding fragment thereof using a cell or host cell described herein (e.g., a cell or a host cell comprising polynucleotides encoding an antibody or antigen-binding fragment thereof described herein). In some aspects, the cell is an isolated cell. In some aspects, exogenous polynucleotides have been introduced into the cell. In some aspects, the method further comprises the step of separating or purifying the antibody or antigen-binding fragment obtained from the cell, host cell, or culture.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies or antigen-binding fragments thereof can be prepared using a wide variety of techniques known in the art including the use of hybridoma, recombinant, and phage display technologies, yeast-based presentation technologies, or a combination thereof. For example, monoclonal antibodies or antigen-binding fragments thereof can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), or as described in Kohler G & Milstein C (1975) Nature 256: 495. Examples of yeast-based presentation methods that can be employed to select and generate the antibodies described herein include those disclosed in, for example, WO2009/036379A2; WO2010/105256; and WO2012/009568, each of which is herein incorporated by reference in its entirety.

In some aspects, a monoclonal antibody or antigen-binding fragment is an antibody or antigen-binding fragment produced by a clonal cell (e.g., hybridoma or host cell producing a recombinant antibody or antigen-binding fragment), wherein the antibody or antigen-binding fragment immunospecifically binds to the spike protein of SARS-CoV-2 as determined, e.g., by ELISA or other antigen-binding assays known in the art, such as lateral flow assays, or in the Examples provided herein. In some aspects, a monoclonal antibody or antigen-binding fragment thereof can be a human antibody or antigen-binding fragment thereof. In some aspects, a monoclonal antibody or antigen-binding fragment thereof can be a Fab fragment or a F(ab′)₂ fragment. Monoclonal antibodies or antigen-binding fragments thereof described herein can, for example, be made by the hybridoma method as described in Kohler G & Milstein C (1975) Nature 256: 495 or can, e.g., be isolated from phage libraries using the techniques as described herein, for example. Other methods for the preparation of clonal cell lines and of monoclonal antibodies and antigen-binding fragments thereof expressed thereby are well known in the art (see, for example, Chapter 11 in: Short Protocols in Molecular Biology, (2002) 5th Ed., Ausubel F M et al., supra).

Antigen-binding fragments of antibodies described herein can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of a tetrameric antibody molecule and contains the complete light chain paired with the VH and CH1 domains of the heavy chain. A F(ab′)₂ fragment contains the two antigen-binding arms of a tetrameric antibody molecule linked by disulfide bonds in the hinge region.

Further, the antibodies or antigen-binding fragments thereof described herein can also be generated using various phage display and/or yeast-based presentation methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody or antigen-binding fragment thereof that binds to a particular antigen can be selected or identified with antigen, e.g., using labeled antigen or antigen bound or captured to a solid surface or bead. Examples of phage display methods that can be used to make the antibodies or fragments described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

### 5.4.1 Polynucleotides

In some aspects, provided herein are polynucleotides comprising a nucleotide sequence encoding a spike protein comprising the amino acid sequence of SEQ ID NO:101.

In some aspects, provided herein are polynucleotides comprising a nucleotide sequence encoding an antibody or antigen-binding fragment thereof described herein or a domain thereof (e.g., a variable light chain region and/or variable heavy chain region) that immunospecifically binds to the spike protein of SARS-CoV-2, and vectors, e.g., vectors comprising such polynucleotides for recombinant expression in host cells (e.g., E. coli and mammalian cells).

In some aspects, provided herein are polynucleotides comprising nucleotide sequences encoding antibodies or antigen-binding fragments thereof, which immunospecifically bind to the spike protein of SARS-CoV-2 and comprise an amino acid sequence as described herein, as well as antibodies or antigen-binding fragments that compete with such antibodies or antigen-binding fragments for binding to SARS-CoV-2 (e.g., in a dose-dependent manner), or which bind to the same epitope as that of such antibodies or antigen-binding fragments.

In some aspects, provided herein are polynucleotides comprising a nucleic acid molecule encoding the heavy chain variable region (VH) listed in Table 5 and/or a nucleic acid molecule encoding the light chain variable region (VL) listed in Tables 6.

TABLE 5
Variable heavy chain nucleotide sequences

| Antibody | VH Nucleotide Sequence (SEQ ID NO) |
|---|---|
| Clone 1 | CAGGTGCAGCTGCAGGAGTCGGGCCCAGGACTGGTGAAGCCTTCGGA |
| | GACCCTGTCCCTCACCTGCACTGTCTCTGGTGGCTCCATCAGTAGTTA |
| | CTACTGGAGCTGGATCCGGCAGCCCCCAGGGAAGGGACTGGAGTGGA |
| | TTGGGTATATCTATTACAGTGGGAGCACCAACTACAACCCCTCCCTCA |
| | AGAGTCGAGTCACCATATCAGTAGACACGTCCAAGAACCAGTTCTCC |
| | CTGAAGCTGAGCTCTGTGACCGCTGCGGACACGGCCGTGTATTACTGT |
| | GCGGGGAGTGGTTATTATACCCATGATGCTTTTGATATCTGGGGCCAA |
| | GGGACAATGGTCACCGTCTCTTCT |
| | (SEQ ID NO: 73) |
| Clone 2 | CAGGTGCAGCTGGTGCAGTCTGGGGCTGAGGTGAAGAAGCCTGGGGC |
| | CTCAGTGAAGGTCTCCTGCAAGGCTTCTGGATACACCTTCACCGGCTA |
| | CTATATGCACTGGGTGCGACAGGCCCCTGGACAAGGGCTTGAGTGGA |
| | TGGGATGGATCAACCCTAACAGTGGTGGCACAAACTATGCACAGAAG |
| | TTTCAGGGCAGGGTCACCATGACCAGGGACACGTCCATCAGCACAGC |
| | CTACATGGAGCTGAGCAGGCTGAGATCTGACGACACGGCCGTGTATT |
| | ACTGTGCGAGAGATTGTGGGAGCTACTACGGGGACTGGTTCGACCCC |
| | TGGGGCCAGGGAACCCTGGTCACCGTCTCCTCT |
| | (SEQ ID NO: 74) |
| Clone 4 | GAGGTGCAGCTGGTGCAGTCTGGAGCAGAGGTGAAAAAGCCGGGGG |
| | AGTCTCTGAAGATCTCCTGTAAGGGTTCTGGATACGGCTTTATCACCT |
| | ACTGGATCGGCTGGGTGCGCCAGATGCCCAGGAAAGGCCTGGAGTGG |
| | ATGGGGATCATCTATCCTGGTGACTCTGAAACCAGATACAGCCCGTCC |
| | TTCCAAGGCCAGGTCACCATCTCAGCCGACAAGTCCATCAACACCGC |
| | CTACCTGCAGTGGAGCAGCCTGAAGGCCTCGGACACCGCCATATATT |
| | ACTGTGCGGGGGGTTCGGGGATTTCTACCCCTATGGACGTCTGGGGCC |
| | AAGGGACCACGGTCACCGTCTCCTCT |
| | (SEQ ID NO: 75) |
| Clone 6 | CAGGTGCAGCTGGTGCAGTCTGGGGCTGAGGTGAAGAAGCCTGGGGC |
| | CTCAGTGAAGGTCTCCTGCAAGGCTTCTGGATACACCTTCACCGGCTA |
| | CTATATGCACTGGGTGCGACAGGCCCCTGGACAAGGGCTTGAGTGGA |
| | TGGGATGGATCAACCCTAACAGTGGTGGCACAAACTATGCACAGAAG |
| | TTTCAGGGCAGGGTCACCATGACCAGGGACACGTCCATCAGCACAGC |
| | CTACATGGAGCTGAGCAGGCTGAGATCTGACGACACGGCCGTGTATT |
| | ACTGTGCGAGAGATTGTGGGAGCTACTACGGGGACTGGTTCGACCCC |
| | TGGGGCCAGGGAACCCTGGTCACCGTCTCCTCT |
| | (SEQ ID NO: 76) |
| Clone 7 | GAAGTGCAGCTGGTGGAGTCTGGGGGAGGCTTGGTACAGCCTGGGGG |
| | GTCCCTGAGACTCTCCTGTGCAGCCTCTGGATTCACCTTCAGTAGCTA |
| | CGACATGCACTGGGTCCGCCAAGCTACAGGAAAAGGTCTGGAGTGGG |
| | TCTCAGCTATTGGTACTGCTGGTGACACATACTATCCAGGCTCCGTGA |
| | AGGGCCGATTCACCATCTCCAGAGAAAATGCCAAGAACTCCTTGTAT |
| | CTTCAAATGAACAGCCTGAGAGCCGGGGACACGGCTGTGTATTACTG |
| | TGCAAGGGGGCACTATGATAGTGGTGGTTATGGGGCTTTTGATATCTG |
| | GGGCCAAGGGACAATGGTCACCGTCTCTTCT |
| | (SEQ ID NO: 77) |
| Clone 9 | CAGGTCACCTTGAAGGAGTCTGGTCCTGTGCTGGTGAAACCCACAGA |
| | GACCCTCACGCTGACCTGCACCGTCTCTGGGTTCTCACTCAGCAATGC |
| | TAGAATGGGTGTGAGCTGGATCCGTCAGCCCCCAGGGAAGGCCCTGG |
| | AGTGGCTTGCACACATTTTTTCGAATGACGAAAAATCCTACAGCACAT |
| | CTCTGAAGAGCAGGCTCACCATCTCCAAGGACACCTCCAAAAGCCAG |
| | GTGGTCCTTACCATGACCAACATGGACCCTGTGGACACAGCCACATAT |
| | TACTGTGCACGGATAATGAGGGAGCTACCCTTTGACTACTGGGGCCA |
| | GGGAACCCTGGTCACCGTCTCCTCT |
| | (SEQ ID NO: 78) |
| Clone 10 | CAGGTGCAGCTGGTGCAGTCTGGGGCTGAGGTGAAGAAGCCTGGGGC |
| | CTCAGTGAAGGTCTCCTGCAAGGCTTCTGGATACACCTTCACCGGCTA |
| | CTATATGCACTGGGTGCGACAGGCCCCTGGACAAGGGCTTGAGTGGA |
| | TGGGATGGATCAACCCTAACAGTGGTGGCACAAACTATGCACAGAAG |
| | TTTCAGGGCAGGGTCACCATGACCAGGGACACGTCCATCAGCACAGT |
| | CTACATGGAGCTGAGCAGGCTGAGATCTGACGACACGGCCGTGTATT |
| | ACTGTGCGAGAGATTGTGGGAGCTACTACGGGGACTGGTTCGACCCC |
| | TGGGGCCAGGGAACCCTGGTCACCGTCTCCTCT |
| | (SEQ ID NO: 79) |
| Clone 11 | GAAGTGCAGCTGGTGGAGTCTGGGGGAGGCTTGGTACAGCCTGGGGG |
| | GTCCCTGAGACTCTCCTGTGCAGCCTCTGGATTCACCTTCAGTAGCTA |
| | CGACATGCACTGGGTCCGCCAAGCTACAGGAAAAGGTCTGGAGTGGG |
| | TCTCAGCTATTGGTACTGCTGGTGACACATACTATCCAGGCTCCGTGA |
| | AGGGCCGATTCACCATCTCCAGAGAAAATGCCAAGAACTCCTTGTAT |
| | CTTCAAATGAACAGCCTGAGAGCCGGGGACACGGCTGTGTATTACTG |
| | TGCAAGGGGGCACTATGATAGTAGTGGTTATGGGGCTTTTGATATCTG |
| | GGGCCAAGGGACAATGGTCACCGTCTCTTCT |
| | (SEQ ID NO: 80) |
| Clone 12 | GAAGTGCAGCTGGTGGAGTCTGGGGGAGGCTTGGTACAGCCTGGGGG |
| | GTCCCTGAGACTCTCCTGTGCAGCCTCTGGATTCACCTTCAGTAGCTA |
| | CGACATGCACTGGGTCCGCCAAGCTACAGGAAAAGGTCTGGAGTGGG |
| | TCTCAGCTATTGGTACTGCTGGTGACACATACTATCCAGGCTCCGTGA |
| | AGGGCCGATTCACCATCTCCAGAGAAAATGCCAAGAACTCCTTGTAT |
| | CTTCAAATGAACAGCCTGAGAGCCGGGGACACGGCTGTGTATTACTG |
| | TGCAAGGGGGCACTATGATAGTGGTGGTTATGGGGCTTTTGATATCTG |
| | GGGCCAAGGGACAATGGTCACCGTCTCTTCT |
| | (SEQ ID NO: 81) |

TABLE 6
Variable light chain nucleotide sequences

| Antibody | VL (kappa) Nucleotide Sequence (SEQ ID NO) |
|---|---|
| Clone 1 | GAAATTGTAATGACACAGTCTCCAGCCACCCTGTCTTTGTCTCCAGGG |
| | GAAAGAGCCACCCTCTCCTGCAGGGCCAGTCAGAGTGTTAGCAGCTA |
| | CTTAGCCTGGTACCAACAGAAACCTGGCCAGCTCCCAGGCTCCTCATC |
| | TATGATGCATCCAACAGGGCCACTGGCATCCCAGCCAGGTTCAGTGG |
| | CAGTGGGTCTGGGACAGACTTCACTCTCACCATCAGCAGCCTAGAGC |
| | CCGAAGACTTTGCAGTTTATTACTGTCAGCAGCGTAGCAACTGGCCTC |
| | TCACTTTCGGCCAAGGGACCAAGGTGGAAATCAA |
| | (SEQ ID NO: 82) |
| Clone 2 | GAAATTGTGTTGACGCAGTCTCCAGGCACCCTGTCTTTGTCTCCAGGG |
| | GAAAGAGCCACCCTCTCCTGCAGGGCCAGTCAGAGTGTTAGCAGCAG |
| | CTACTTAGCCTGGTACCAGCAGAAACCTGGCCAGGCTCCCAGGCTCCT |
| | CATCTATGGTGCATCCAGCAGGGCCACTGGCATCCCAGACAGGTTCA |
| | GTGGCAGTGGGTCTGGGACAGACTTCACTCTCACCATCAGCAGACTG |
| | GAGCCTGAAGATTTTGCAGTGTATTACTGTCAGCAGTATGGTAGCTCA |
| | CCTCTCACTTTTGGCCAAGGGACCAAGGTGGAAATCAAA |
| | (SEQ ID NO: 83) |
| Clone 4 | GACATCCAGTTGACCCAGTCTCCAGACTCCCTGGCTGTGTCTCTGGGC |
| | GAGAGGGCCACCATCAACTGCAAGTCCAGCCAGAGTGTTTTATACAG |
| | CTCCATCAATAAGAACTACTTAGCTTGGTACCAGCAGAAACCAGGAC |
| | AGCCTCCTAAGCTGCTCATTTACTGGGCATCTACCCGGGAATCCGGGG |
| | TCCCTGACCGATTCAGTGGCAGCGGGTCTGGCACAGATTTCACTCTCA |
| | CCATCAGCAGCCTGCAGGCTGAAGATGTGGCAGTTTATTACTGTCAGC |
| | AATATTATAGTACTCCGTACACTTTCGGCCAAGGGACACGACTGGAG |
| | ATTAAA |
| | (SEQ ID NO: 84) |
| Clone 6 | GAAATTGTGTTGACGCAGTCTCCAGGCACCCTGTCTTTGTCTCCAGGG |
| | GAAAGAGCCACCCTCTCCTGCAGGGCCAGTCAGAGTGTTAGCAGCAG |
| | CTACTTAGCCTGGTACCAGCAGAAACCTGGCCAGGCTCCCAGGCTCCT |
| | CATCTATGGTGCATCCAGCAGGGCCACTGGCATCCCAGACAGGTTCA |
| | GTGGCAGTGGGTCTGGGACAAACTTCACTCTCACCATCAGCAGACTG |
| | GAGCCTGAAGATTTTGCAGTGTATTACTGTCAGCAGTATGGTAGCTCA |
| | CCTCTCACGTTCGGCCAAGGGACCAAGGTGGAAATCAAA |
| | (SEQ ID NO: 85) |
| Clone 7 | GACATCCAGCTGACCCAGTCTCCATCCTTCCTGTCTGCATCTGTAGGA |
| | GACAGAGTCACCATCACTTGCCGGGCCAGTCAGGGCATTAGCAGTTA |
| | TTTAGCCTGGTATCAGCAAAAACCAGGGAAAGCCCCTAAGCTCCTGA |
| | TCTATACTGCATCCACTTTGCAAAGTGGGGTCCCATCAAGGTTCAGCG |
| | GCAGTGGATCTGGGACAGAATTCACTCTCACAATCAGCAGCCTGCAG |
| | CCTGAAGATTTTGCAACTTATTACTGTCAACAGTATTATAGTTACCCTC |
| | CGCTCACTTTCGGCCCTGGGACCAAAGTGGATATCAAA |
| | (SEQ ID NO: 86) |
| Clone 9 | GACATCCAGATGACCCAGTCTCCTTCCACCCTGTCTGCATCTGTAGGA |
| | GACAGAGTCACCATCACTTGCCGGGCCAGTCAGAGTATTAGTAGCTG |
| | GTTGGCCTGGTATCAGCAGAAACCAGGGAAAGCCCCTAAGCTCCTGA |
| | TCTATAAGGCGTCTAGTTTAGAAAGTGGGGTCCCATCAAGGTTCAGCG |
| | GCAGTGGATCTGGGACAGAATTCACTCTCACCATCAGCAGCCTGCAG |
| | CCTGATGATTTTGCAACTTATTACTGCCAACAGTATAATAGTTATTCGT |
| | GGACGTTCGGCCAAGGGACCAAGGTGGAAATCAAA |
| | (SEQ ID NO: 87) |
| Clone 10 | GAAATTGTGTTGACGCAGTCTCCAGGCACCCTGTCTTTGTCTCCAGGG |
| | GAAAGAGCCACCCTCTCCTGCAGGGCCAGTCAGAGTGTTAGCAGCAG |
| | CTACTTAGCCTGGTACCAGCAGAAACCTGGCCAGGCTCCCAGGCTCCT |
| | CATCTATGGTGCATCCAGCAGGGCCACTGGCATCCCAGACAGGTTCA |
| | GTGGCAGTGGGTCTGGGACAGACTTCACTCTCACCATCAGCAGACTG |
| | GAGCCTGAAGATTTTGCAGTGTATTACTGTCAGCAGTATGGTAGCTCA |
| | CCTCTCACTTTCGGCGGAGGGACCAAGGTGGAGATCAAA |
| | (SEQ ID NO: 88) |
| Clone 11 | GCCATCCGGATGACCCAGTCTCCATCCTCATTCTCTGCATCTACAGGA |
| | GACAGAGTCACCATCACTTGTCGGGCGAGTCAGGGTATTAGCAGTTAT |
| | TTAGCCTGGTATCAGCAAAAACCAGGGAAAGCCCCTAAGCTCCTGAT |
| | CTATGCTGCATCCACTTTGCAAAGTGGGGTCCCATCAAGGTTCAGCGG |
| | CAGTGGATCTGGGACAGATTTCACTCTCACCATCAGCTGCCTGCAGTC |
| | TGAAGATTTTGCAACTTATTACTGTCAACAGTATTATAGTTACCCTCC |
| | GCTCACTTTCGGCGGAGGGACCAAGGTGGAGATCAAA |
| | (SEQ ID NO: 89) |
| Clone 12 | GACATCCAGTTGACCCAGTCTCCATCCTTCCTGTCTGCATCTGTAGGA |
| | GACAGAGTCACCATCACTTGCCGGGCCAGTCAGGGCATTAGCAGTTA |
| | TTTAGCCTGGTATCAGCAAAAACCAGGGAAAGCCCCTAAGCTCCTGA |
| | TCTATACTGCATCCACTTTGCAAAGTGGGGTCCCATCAAGGTTCAGCG |
| | GCAGTGGATCTGGGACAGAATTCACTCTCACCATCAGCTGCCTGCAGT |
| | CTGAAGATTTTGCAACTTATTACTGTCAACAGTATTATAGTTACCCTCC |
| | GCTCACTTTCGGCCAGGGGACCAAGGTGGAGATCAAA |
| | (SEQ ID NO: 90) |

Also provided herein are polynucleotides encoding a spike protein of SEQ ID NO:101 or encoding an antibody or antigen-binding fragment thereof described herein that specifically binds to the spike protein of SARS-CoV-2 that are optimized, e.g., by codon/RNA optimization, replacement with heterologous signal sequences, and elimination of mRNA instability elements. Methods to generate optimized nucleic acids for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

A polynucleotide encoding an antibody or antigen-binding fragment thereof described herein or a domain thereof can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody or antigen-binding fragment thereof. Such PCR amplification methods can be used to obtain nucleic acids comprising the sequence encoding the variable light chain region and/or the variable heavy chain region of an antibody or antigen-binding fragment thereof. The amplified nucleic acids can be cloned into vectors for expression in host cells and for further cloning, for example, to generate chimeric and humanized antibodies or antigen-binding fragments thereof.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure. In some aspects, a polynucleotide is purified from natural components.

### 5.4.2 Cells and Vectors

In some aspects, provided herein are vectors (e.g., expression vectors) comprising polynucleotides comprising nucleotide sequences encoding a spike protein comprising the amino acid sequence of SEQ ID NO:101 for recombinant expression in host cells, e.g., in mammalian cells. Also provided herein are cells, e.g. host cells, comprising such vectors for recombinantly expressing spike proteins or trimers of spike proteins. In a particular aspect, provided herein are methods for producing a spike protein comprising the amino acid sequence of SEQ ID NO:101 or trimer thereof, comprising expressing such protein in a host cell.

In some aspects, provided herein are vectors (e.g., expression vectors) comprising polynucleotides comprising nucleotide sequences encoding antibodies and antigen-binding fragments thereof or a domain thereof that bind the spike for recombinant expression in host cells, e.g., in mammalian cells. Also provided herein are cells, e.g. host cells, comprising such vectors for recombinantly expressing antibodies or antigen-binding fragments thereof described herein (e.g., human antibodies or antigen-binding fragments thereof) that bind to the spike. In a particular aspect, provided herein are methods for producing an antibody or antigen-binding fragments thereof described herein, comprising expressing such antibody or antigen-binding fragment thereof in a host cell.

In some aspects, recombinant expression a spike protein comprising the amino acid sequence of SEQ ID NO:101, involves construction of an expression vector containing a polynucleotide that encodes the spike protein. Once a polynucleotide encoding a spike protein comprising the amino acid sequence of SEQ ID NO:101 has been obtained, the vector for the production of the protein thereof can be produced by recombinant DNA technology using techniques well known in the art. Thus, methods for preparing a protein by expressing a polynucleotide containing a spike protein-encoding nucleotide sequence are described herein.

In some aspects, recombinant expression of an antibody or antigen-binding fragment thereof or domain thereof described herein (e.g., a heavy or light chain described herein) that specifically binds to the spike involves construction of an expression vector containing a polynucleotide that encodes the antibody or antigen-binding fragment thereof or domain thereof. Once a polynucleotide encoding an antibody or antigen-binding fragment thereof or domain thereof (e.g., heavy or light chain variable domain) described herein has been obtained, the vector for the production of the antibody or antigen-binding fragment thereof can be produced by recombinant DNA technology using techniques well known in the art. Thus, methods for preparing a protein by expressing a polynucleotide containing an antibody or antigen-binding fragment thereof or domain thereof (e.g., light chain or heavy chain) encoding nucleotide sequence are described herein.

Methods which are well known to those skilled in the art can be used to construct expression vectors containing protein- or antibody or antigen-binding fragment thereof or domain thereof (e.g., light chain or heavy chain)-coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a protein or antibody or antigen-binding fragment thereof described herein, a heavy or light chain, a heavy or light chain variable domain, or a heavy or light chain CDR, operably linked to a promoter. Such vectors can, for example, include the nucleotide sequence encoding the constant region of the antibody or antigen-binding fragment thereof (see, e.g., International Publication Nos. WO 86/05807 and WO 89/01036; and U.S. Pat. No. 5,122,464), and variable domains of the antibody or antigen-binding fragment thereof can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce a protein or an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of an antibody provided in Tables 1-4) or a domain thereof (e.g., the VH, the VL, the VH and the VL, the heavy chain, or the light chain of an antibody provided in Tables 3 and 4). Thus, provided herein are host cells containing a polynucleotide encoding a protein or an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of antibody provided in Tables 1-4) or a domain thereof (e.g., the VH, the VL, the VH and the VL, the heavy chain, or the light chain of antibody provided in Tables 3 and 4), operably linked to a promoter for expression of such sequences in the host cell. In some aspects, for the expression of double-chained antibodies or antigen-binding fragments thereof, vectors encoding both the heavy and light chains, individually, can be co-expressed in the host cell for expression of the entire immunoglobulin, as detailed below. In some aspects, a host cell contains a vector comprising a polynucleotide encoding both the heavy chain and light chain of an antibody described herein (e.g., the heavy and the light chain of antibody provided in Tables 1-4), or a domain thereof (e.g., the VH and the VL of antibody provided in Tables 3-4). In some aspects, a host cell contains two different vectors, a first vector comprising a polynucleotide encoding a heavy chain or a heavy chain variable region of an antibody or antigen-binding fragment thereof described herein, and a second vector comprising a polynucleotide encoding a light chain or a light chain variable region of an antibody described herein (e.g., an antibody comprising the six CDRs of an antibody provided in Tables 1 and 2), or a domain thereof. In some aspects, a first host cell comprises a first vector comprising a polynucleotide encoding a heavy chain or a heavy chain variable region of an antibody or antigen-binding fragment thereof described herein, and a second host cell comprises a second vector comprising a polynucleotide encoding a light chain or a light chain variable region of an antibody or antigen-binding fragment thereof described herein (e.g., an antibody or antigen-binding fragment thereof comprising the six CDRs of an antibody provided in Tables 1 and 2). In some aspects, a heavy chain/heavy chain variable region expressed by a first cell associated with a light chain/light chain variable region of a second cell to form an antibody or antigen-binding fragment thereof described herein (e.g., antibody or antigen-binding fragment thereof comprising the six CDRs of an antibody provided in Tables 1 and 2). In some aspects, provided herein is a population of host cells comprising such first host cell and such second host cell.

In some aspects, provided herein is a population of vectors comprising a first vector comprising a polynucleotide encoding a light chain/light chain variable region of an antibody or antigen-binding fragment thereof described herein, and a second vector comprising a polynucleotide encoding a heavy chain/heavy chain variable region of an antibody or antigen-binding fragment thereof described herein (e.g., antibody or antigen-binding fragment thereof comprising the CDRs of an antibody provided in Table 1 and 2). Alternatively, a single vector can be used which encodes, and is capable of expressing, both heavy and light chain polypeptides.

In some aspects, provided herein is a vector comprising a polynucleotide encoding a spike protein comprising the amino acid sequence of SEQ ID NO:101, e.g., for recombinant expression in host cells, e.g., in mammalian cells.

A variety of host-expression vector systems can be utilized to express proteins and antibodies and antigen-binding fragments thereof described herein (e.g., an antibody or antigen-binding fragment thereof comprising the CDRs of an antibody provided in Tables 1 and 2) (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express a protein or an antibody or antigen-binding fragment thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing proteins or antibodies and antigen-binding fragments thereof described herein (e.g., an antibody or antigen-binding fragment thereof comprising the CDRs of an antibody provided in Table 1) are CHO cells, for example CHO cells from the CHO GS System™ (Lonza). In some aspects, cells for expressing proteins or antibodies and antigen-binding fragments thereof described herein are human cells, e.g., human cell lines. In some aspects, a mammalian expression vector is pOptiVEC™ or pcDNA3.3. In some aspects, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In some aspects, proteins or antibodies or antigen-binding fragments thereof described herein are produced by CHO cells or NS0 cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In some aspects, antibodies or antigen-binding fragments thereof described herein that specifically bind to the spike protein of SARS-CoV-2 are produced in mammalian cells, such as CHO cells.

Once a protein or an antibody or antigen-binding fragment thereof described herein has been produced by recombinant expression, it can be purified by any method known in the art for purification of a protein or an immunoglobulin molecule, for example, by chromatography (e.g., ion exchange, affinity, particularly by affinity for the specific antigen after Protein A, and size exclusion chromatography), centrifugation, differential solubility, or by any other standard technique for the purification of proteins. Further, the proteins or antibodies or antigen-binding fragments thereof described herein can be fused to heterologous polypeptide sequences described herein or otherwise known in the art to facilitate purification.

In some aspects, a protein (e.g., a spike protein comprising the amino acid sequence set forth in SEQ ID NO:101) or an antibody or antigen-binding fragment thereof described herein is isolated or purified. Generally, an isolated protein or antibody or antigen-binding fragment thereof is one that is substantially free of other proteins or antibodies or antigen-binding fragments thereof with different antigenic specificities than the isolated antibody or antigen-binding fragment thereof. For example, in some aspects, a preparation of a protein (e.g., a spike protein comprising the amino acid sequence set forth in SEQ ID NO:101) or an antibody or antigen-binding fragment thereof described herein is substantially free of cellular material and/or chemical precursors.

### 5.5 Detection & Diagnostic Uses

A protein (e.g., a spike protein comprising the amino acid sequence set forth in SEQ ID NO:101) (see e.g., Section 6.2) and/or an antibody or antigen-binding fragment thereof that binds to the spike protein of SARS-CoV-2 described herein (see, e.g., Section 6.3) can be used to assay protein levels of anti-SARS-CoV-2 antibodies in a biological sample using classical methods known to those of skill in the art, including immunoassays, such as the enzyme linked immunosorbent assay (ELISA). Suitable antibody assay labels are known in the art and include enzyme labels, such as, glucose oxidase. Such labels can be used to label an antibody or antigen-binding fragment thereof described herein or to labels antibodies or antigen-binding fragments thereof in a test sample (e.g., a sample obtained from a subject). Alternatively, a second antibody or antigen-binding fragment thereof that recognizes an antibody or antigen-binding fragment thereof that binds to the spike protein of SARS-CoV-2 described herein or that recognizes antibodies or antigen-binding fragments thereof in a test sample (e.g., a sample obtained from a subject) can be labeled and used to detect anti-SARS-CoV-2 antibodies.

Assaying for anti-SARS-CoV-2 antibodies includes qualitatively or quantitatively measuring or estimating anti-SARS-CoV-2 antibodies in a biological sample either directly (e.g., by determining or estimating absolute protein level) or relatively (e.g., by comparing to the level in a second biological sample).

As used herein, the term “biological sample” refers to any biological sample obtained from a subject, cell line, tissue, or other source potentially containing anti-SARS-CoV-2 antibodies. Methods for obtaining tissue biopsies and body fluids from animals (e.g., humans) are well known in the art.

Antibodies or antigen-binding fragments thereof that bind to the spike protein of SARS-CoV-2 described herein can carry a detectable or functional label. When fluorescence labels are used, currently available microscopy and fluorescence-activated cell sorter analysis (FACS) or combination of both methods procedures known in the art may be utilized to identify and to quantitate the specific binding members. Antibodies or antigen-binding fragments thereof that bind to the spike protein of SARS-CoV-2 described herein can carry a fluorescence label. Exemplary fluorescence labels include, for example, reactive and conjugated probes, e.g., Aminocoumarin, Fluorescein and Texas red, Alexa Fluor dyes, Cy dyes and DyLight dyes. An antibody or antigen-binding fragment thereof that specifically binds to the spike protein of SARS-CoV-2 can carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹In, ¹¹⁷Lu, ¹²¹I, ¹²⁴I, ¹²⁵I, ¹³¹I, ¹⁹⁸Au, ²¹¹At, ²¹³Bi, ²²⁵Ac and ¹⁸⁶Re. When radioactive labels are used, currently available counting procedures known in the art may be utilized to identify and quantitate the specific binding of an antibodies or antigen-binding fragment thereof that binds to the spike protein of SARS-CoV-2. In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an antibody or antigen-binding fragment thereof that binds to the spike protein of SARS-CoV-2 under conditions that allow for the formation of a complex between the antibody or antigen-binding fragment thereof and the spike protein of SARS-CoV-2. Any complexes formed between the antibodies or antigen-binding fragments and the spike proteins of SARS-CoV-2 are detected and compared in the sample (and optionally a control). In light of the specific binding of the antibodies or antigen-binding fragments thereof that bind to the spike protein of SARS-CoV-2 described herein for SARS-CoV-2, the antibodies or antigen-binding fragments thereof can be used to specifically detect anti-SARS-CoV-2 antibodies or antigen-binding fragments thereof (e.g., in a subject).

In some aspects, the subject has been exposed to SARS-CoV-2. In some aspects, the subject has not been exposed to SARS-CoV-2. In some aspects, the subject is at risk of exposure to SARS-CoV-2.

Also included herein is an assay system that can be prepared in the form of a test kit for the quantitative analysis of the extent of the presence of, for instance, anti-SARS-CoV-2 antibodies or antigen-binding fragments thereof. The system or test kit may comprise a labeled component, e.g., a labeled antibody or antigen-binding fragment, and one or more additional immunochemical reagents. See, e.g., Section 6.6 below for more on kits.

In some aspects, provided herein are methods for in vitro detecting anti-SARS-CoV-2 antibodies or antigen-binding fragments thereof in a sample, comprising contacting the sample with a spike protein comprising the amino acid sequence set forth in SEQ ID NO:101 or a trimer comprising spike proteins comprising the amino acid sequences set forth in SEQ ID NO:101. An antibody or antigen-binding fragment thereof described herein may also be used in the assay, e.g., as a positive control.

In some aspects, provided herein is a spike protein comprising the amino acid sequence set forth in SEQ ID NO:101 or a trimer comprising spike proteins comprising the amino acid sequences set forth in SEQ ID NO:101 for use in detecting (e.g., in vitro) anti-SARS-CoV-2 antibodies or antigen-binding fragments thereof in a sample. The detecting can be performed using an assay that further comprises the use of an antibody or antigen-binding fragment thereof described herein, e.g., as a positive control.

In one aspect, provided herein is a spike protein comprising the amino acid sequence set forth in SEQ ID NO:101 or a trimer comprising spike proteins comprising the amino acid sequences set forth in SEQ ID NO:101 for use as a diagnostic. The detecting can be performed using an assay that further comprises the use of an antibody or antigen-binding fragment thereof described herein, e.g., as a positive control.

In some aspects, the subject is a human.

An assay for detecting anti-SARS-CoV-2 antibodies or antigen-binding fragments thereof in a sample using a spike protein comprising the amino acid sequence set forth in SEQ ID NO:101, a trimer comprising spike proteins comprising the amino acid sequence set forth in SEQ ID NO:101, and/or an antibody or antigen-binding fragment thereof provided herein can comprise contacting a spike protein or a spike protein trimer (optionally wherein the spike protein comprising the amino acid sequence set forth in SEQ ID NO:101 or wherein the trimer comprises spike proteins comprising the amino acid sequence set forth in SEQ ID NO:101) with a test sample. The test sample can be a sample obtained from a patient. The test sample can be a sample obtained from a bodily flued. The test sample can be a composition containing antibodies and/or antigen-binding fragments thereof obtained from a subject. The test sample can contain IgG antibodies or antigen-binding fragments thereof. The test sample can contain IgM antibodies or antigen-binding fragments thereof. The test sample can contain IgG or IgM antibodies or antigen-binding fragments thereof. The assay can further comprise detecting binding of any antibodies or antigen-binding fragments thereof in the sample to the spike protein and/or the trimer.

In some aspects, detecting the binding of any antibodies or antigen-binding fragments thereof in the sample to the spike protein and/or the trimer comprises the use of a secondary antibody or antigen-binding fragment thereof. The secondary antibody or antigen-binding fragment thereof can be an antibody or antigen-binding fragment thereof that binds to the Fc region of antibodies or antigen-binding fragments thereof. The secondary antibody or antigen-binding fragment thereof can be labeled with a detectable label, e.g., a detectable label provided herein. The presence of the detectable label can indicate the presence of an antibody or antigen-binding fragment in the test sample that binds to the spike protein and/or the trimer.

In some aspects, detecting the binding of any antibodies or antigen-binding fragments thereof in the sample to the spike protein and/or the trimer comprises the use of an antibody or antigen-binding fragment thereof provided herein, e.g., as a control. In such cases a secondary antibody or antigen-binding fragment thereof (optionally labeled) can bind to the antibody or antigen-binding fragment thereof provided herein and can bind to an antibody or antigen-binding fragment in the sample.

### 5.6 Kits

Provided herein are kits comprising one or more proteins, trimers, or antibodies or antigen-binding fragments thereof described herein or conjugates thereof.

Also provided herein are kits that can be used in diagnostic methods. In some aspects, a kit comprises a spike protein comprising the amino acid sequence of SEQ ID NO:101 or a timer comprising proteins comprising the amino acid sequence of SEQ ID NO:101, in one or more containers. In some aspects, a kit comprises an antibody or antigen-binding fragment thereof described herein, preferably a purified antibody or antigen-binding fragment thereof, in one or more containers. In some aspects, kits described herein contain a substantially isolated SARS-CoV-2 spike protein or trimer provided herein and/or anti-SARS-CoV-2 antibodies or antigen-binding fragments thereof provided herein, e.g. that can be used as a control. In some aspects, the kits described herein further comprise a control antibody or antigen-binding fragment thereof which does not react with a SARS-CoV-2 spike protein antigen. In some aspects, kits described herein contain one or more elements for detecting the binding of an antibody or antigen-binding fragment thereof to a SARS-CoV-2 spike protein and an an anti-SARS-CoV-2 antibody or antigen-binding fragment thereof (e.g., the antibody or antigen-binding fragment thereof can be conjugated to a detectable substrate such as a fluorescent compound, an enzymatic substrate, a radioactive compound or a luminescent compound, or a second antibody or antigen-binding fragment thereof which recognizes the first antibody or antigen-binding fragment thereof can be conjugated to a detectable substrate). In some aspects, a kit provided herein can include a recombinantly produced or chemically synthesized SARS-CoV-2 spike protein and/or a SARS-CoV-2 spike protein trimer. The SARS-CoV-2 spike protein and/or trimer provided in the kit can also be attached to a solid support. In some aspects, the detecting means of the above described kit includes a solid support to which a SARS-CoV-2 spike protein or trimer is attached. Such a kit can also include a non-attached reporter-labeled anti-human antibody or antigen-binding fragment thereof or anti-mouse/rat antibody or antigen-binding fragment thereof. In this aspect, binding of the antibody or antigen-binding fragment thereof that binds to the spike protein of SARS-CoV-2 to the SARS-CoV-2 spike protein antigen can be detected by binding of the reporter-labeled antibody or antigen-binding fragment thereof.

The following examples are offered by way of illustration and not by way of limitation.

### 6. EXAMPLES

The examples in this Examples Section (i.e., Section 6) are offered by way of illustration, and not by way of limitation.

### 6.1 Example 1: Antibodies that Bind to the Spike Protein of SARS-CoV-2

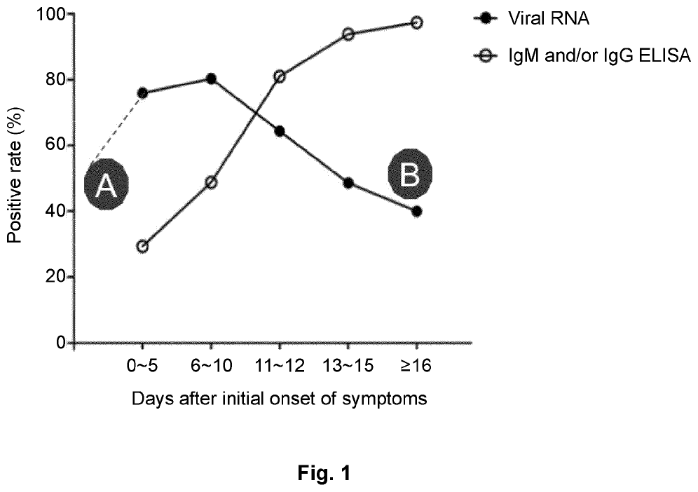

PCR tests that determine the presence or absence of SARS-CoV-2 in a person are available. A negative PCR test result indicates that a person was not infected at the time the sample used in the PCT test was obtained. However, it does not differentiate between people who have not been infected (and therefore may be infected and infectious in the future) and people who have previously been infected and have now recovered (and therefore may not be infectious in the future). Assays that test for the presence of anti-SARS-CoV-2 in a person can differentiate between these two types of people. FIG. 1 shows the dynamics of the positive rate of viral RNA and antibody in patients at different stages of disease. Early in disease (e.g., 0-5 days after the initial onset of symptoms), a person may be more likely to test positive for SARS-CoV-2 in a PCR assay and less likely to have anti-SARS-CoV-2 antibodies. Later in disease (e.g., more than 15 days after the initial onset of symptoms), a person may no longer test positive for SARS-CoV-2 in a PCR assay, but may have developed anti-SARS-CoV-2 antibodies. Accordingly, assays for determining the presence of anti-SARS-CoV-2 antibodies were developed.

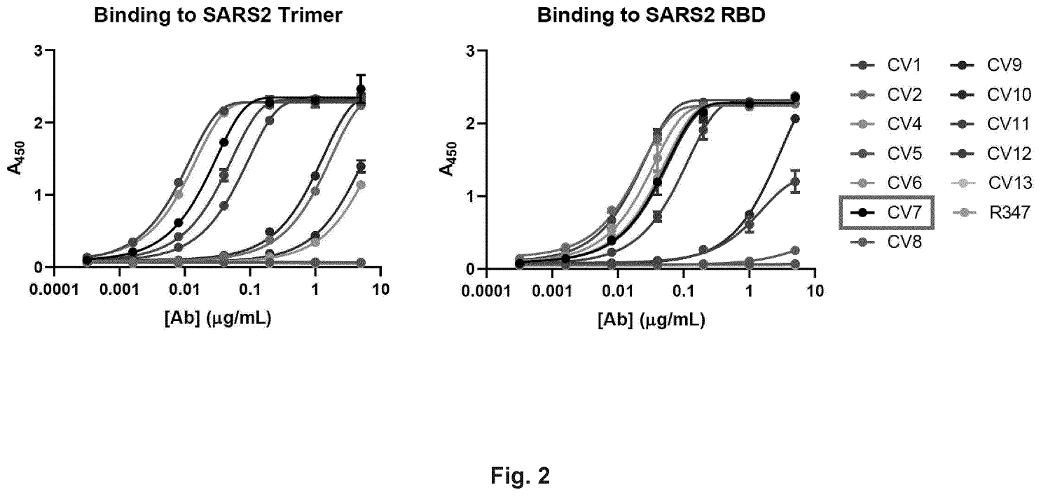

The assays focus on the spike protein of SARS-CoV-2 because antibodies that bind to the spike protein of SARS-CoV-2 can neutralize the virus. Later Flow Tests (LFTs) and Enzyme Linked Immunosorbent Assays (ELISAs) were developed. In order to test the performance of the LFT, anti-SARS-CoV-2 antibodies were produced as positive and negative controls. The antibodies, CV1-CV13 (sequences provided in Tables 1-4), were assayed for their ability to bind to a SARS2 trimer and to the receptor binding domain (RBD) of SARS-CoV-2. The results are shown in FIG. 2. Antibody CV7 is particularly effective in binding to both the trimer and the RBD.

### 6.2 Example 2: Antibodies do not Neutralize SARS-CoV-2 Pseudovirus

The antibodies were also assayed for their ability to neutralize SARS-CoV-2 pseudovirus.

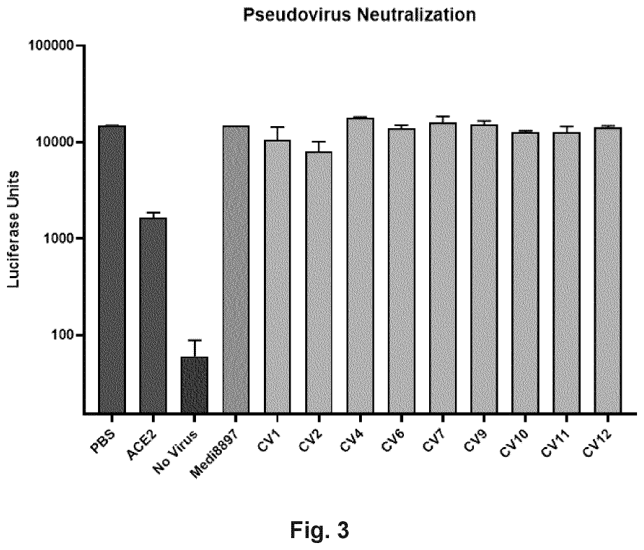

FIG. 3 demonstrates that the CV1, CV2, CV4, CV6, CV7, CV9, CV10, CV11, and CV12 antibodies do not neutralize SARS-CoV-2 pseudovirus.

### 6.3 Example 3: Antibodies are Effective in Lateral Flow Tests

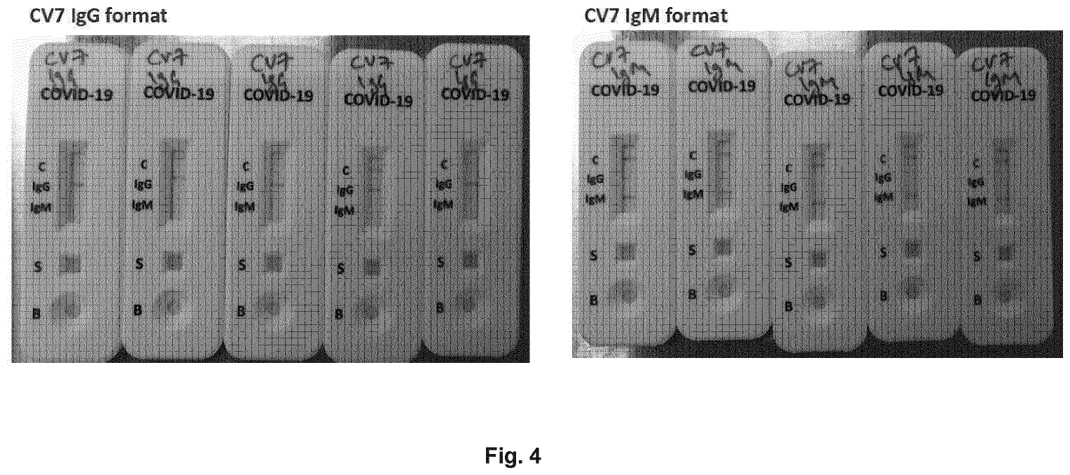

CV7 was expressed in an IgG format and in an IgM format, both of which were tested in LFTs. As shown in FIG. 4, the LFT consistently produced accurate results using both CV7 formats.

### 6.4 Example 4: Antibodies are Effective in Enzyme Linked Immunosorbent Assays

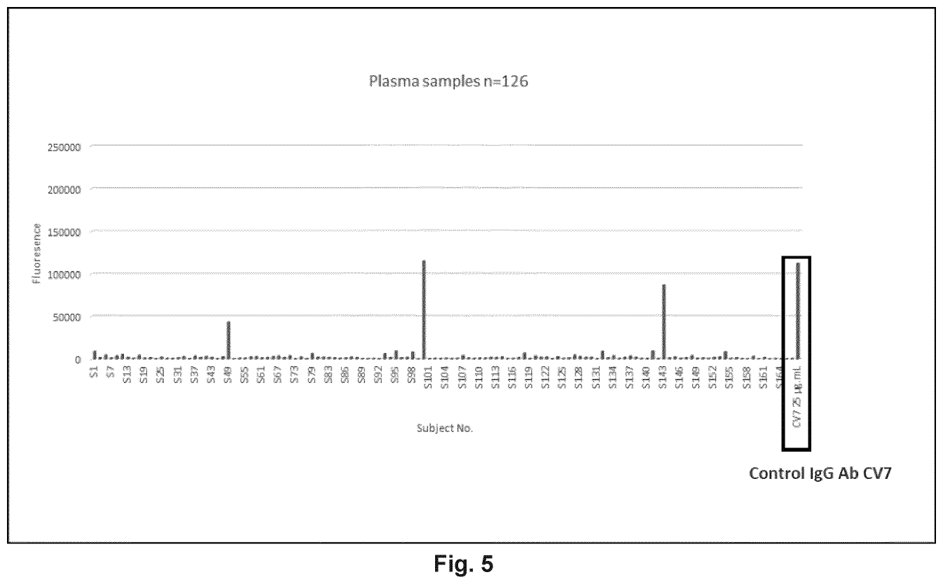

In order to test the specificity of the assay, 126 plasma samples that were taken from healthy volunteers before December of 2019 (pre-pandemic samples) were tested in the ELISA assay. The results are shown in FIG. 5. The specificity of the assay was 98%, and the sensitivity of the assay was 100%.

### 6.5 Example 5: Assays for Detection of Anti-SARS-CoV-2 Antibodies

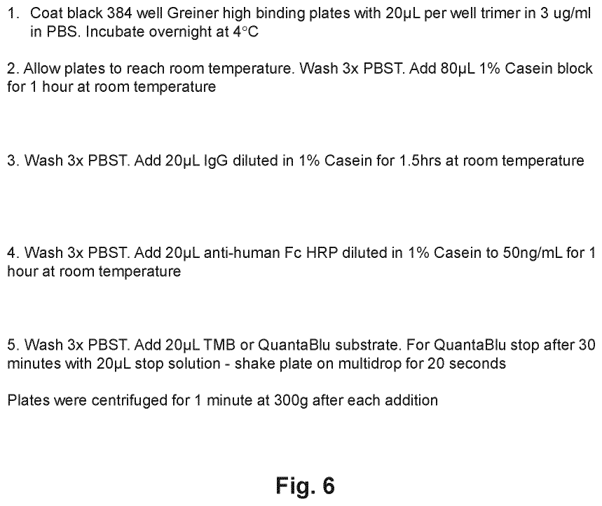

An exemplary assay for detecting anti-SARS-CoV-2 antibodies or antigen-binding fragments thereof is provided in FIG. 6. In such an exemplary assay, black 384-well Greiner high binding plates are coated with 20 μl per well of a spike protein trimer comprising the amino acid sequence of SEQ ID NO:101 in 3 μg/ml of PBS. The plates are incubated overnight at 4° C. The plates are then allowed to reach room temperature and washed three times with PB ST. Then 80 μl of 1% Casein block is added for 1 hour at room temperature, after which the plates are washed three times with PBST. Then 20 μl of an test sample (e.g., an IgG test sample) diluted in 1% Casein block is added for 1.5 hours at room temperature. The test sample can be a sample obtained from a patient (e.g., a human patient.) As a control, a similar 20 μl sample containing a control antibody (e.g., CV07) can be added to a well. The plates are then washed three times with PBST. Then 20 μl of anti-human Fc HRP diluted in 1% Casein to 50 ng/ml is added for 1 hour at room temperature, and the plates are again washed three times with PBST. Then 20 μl TMB or QuantaBlu substrate is added. For QuantaBlue, 20 μl of stop solution is added after 30 minutes to stop the reaction, and the plate is shaken on multidrop for 20 seconds. Throughout the protocol, plates are centrifuged for 1 minute at 300 g after each addition. The presence of HRP in a well (e.g., at a level similar to the level of HRP in a well with a control antibody such as CV07) indicates that the sample in that well contained antibody or antigen-binding fragment thereof binds to the spike protein trimer of SARS-CoV-2.

What is claimed:

1. An antibody or antigen-binding fragment thereof that specifically binds to the same epitope of the spike protein of SARS-CoV-2 as an antibody comprising a variable heavy chain (VH) and a variable light chain (VL) wherein the amino acid sequences of the VH and VL comprise the sequences of:
(a) SEQ ID NO: 55 and SEQ ID NO: 64, respectively;
(b) SEQ ID NO: 56 and SEQ ID NO: 65, respectively;
(c) SEQ ID NO: 57 and SEQ ID NO: 66, respectively;
(d) SEQ ID NO: 58 and SEQ ID NO: 67, respectively;
(e) SEQ ID NO: 59 and SEQ ID NO: 68, respectively;
(f) SEQ ID NO: 60 and SEQ ID NO: 69, respectively;
(g) SEQ ID NO: 61 and SEQ ID NO: 70, respectively;
(h) SEQ ID NO:62 and SEQ ID NO: 71, respectively; or
(i) SEQ ID NO: 63 and SEQ ID NO: 72, respectively.

2. An antibody or antigen-binding fragment thereof that competitively inhibits binding of a reference antibody to the spike protein of SARS-CoV-2, wherein the reference antibody comprises a variable heavy chain (VH) and a variable light chain (VL), wherein the amino acid sequences of the VH and VL comprise the sequences of:
(a) SEQ ID NO: 55 and SEQ ID NO: 64, respectively;
(b) SEQ ID NO: 56 and SEQ ID NO: 65, respectively;
(c) SEQ ID NO: 57 and SEQ ID NO: 66, respectively;
(d) SEQ ID NO: 58 and SEQ ID NO: 67, respectively;
(e) SEQ ID NO: 59 and SEQ ID NO: 68, respectively;
(f) SEQ ID NO: 60 and SEQ ID NO: 69, respectively;
(g) SEQ ID NO: 61 and SEQ ID NO: 70, respectively;
(h) SEQ ID NO:62 and SEQ ID NO: 71, respectively; or
(i) SEQ ID NO: 63 and SEQ ID NO: 72, respectively.

3. An antibody or antigen-binding fragment thereof that specifically binds to the spike protein of SARS-CoV-2, comprising the VH-CDR1-3 and VL CDR1-3 amino acid sequences selected from the group consisting of:
(a) SEQ ID NOs: 1, 2, and 3 and SEQ ID NOs: 28, 29, and 30, respectively;
(b) SEQ ID NOs: 4, 5, and 6 and SEQ ID NOs: 31, 32, and 33, respectively;
(c) SEQ ID NOs: 7, 8, and 9 and SEQ ID NOs: 34, 35, and 36, respectively;
(d) SEQ ID NOs: 10, 11, and 12 and SEQ ID NOs: 37, 38, and 39, respectively;
(e) SEQ ID NOs: 13, 14, and 15 and SEQ ID NOs: 40, 41, and 42, respectively;
(f) SEQ ID NOs: 16, 17, and 18 and SEQ ID NOs: 43, 44, and 45, respectively;
(g) SEQ ID NOs: 19, 20, and 21 and SEQ ID NOs: 46, 47, and 48, respectively;
(h) SEQ ID NOs: 22, 23, and 24 and SEQ ID NOs: 49, 50, and 51, respectively; or
(i) SEQ ID NOs: 25, 26, and 27 and SEQ ID NOs: 52, 53, and 54, respectively.

4. The antibody or antigen-binding fragment thereof of any one of claims 1-3, comprising a variable heavy chain (VH) and a variable light chain (VL), wherein the variable heavy chain (VH) amino acid sequence is selected from the group consisting of:
(a) SEQ ID NO: 55;
(b) SEQ ID NO: 56;
(c) SEQ ID NO: 57;
(d) SEQ ID NO: 58;
(e) SEQ ID NO: 59;
(f) SEQ ID NO: 60;
(g) SEQ ID NO: 61;
(h) SEQ ID NO:62; and
(i) SEQ ID NO: 63.

5. The antibody or antigen-binding fragment thereof of any one of claims 1-4, comprising a variable heavy chain (VH) and a variable light chain (VL), wherein the variable light chain (VL) amino acid sequence selected from the group consisting of:
(a) SEQ ID NO: 64;
(b) SEQ ID NO: 65;
(c) SEQ ID NO: 66;
(d) SEQ ID NO: 67;
(e) SEQ ID NO: 68;
(f) SEQ ID NO: 69;
(g) SEQ ID NO: 70;
(h) SEQ ID NO: 71; and
(i) SEQ ID NO: 72.

6. An antibody or antigen-binding fragment thereof that specifically binds to the spike protein of SARS-CoV-2 comprising a variable heavy chain (VH) and a variable light chain (VL), wherein the variable heavy chain (VH) amino acid sequence is selected from the group consisting of:
(a) SEQ ID NO: 55;
(b) SEQ ID NO: 56;
(c) SEQ ID NO: 57;
(d) SEQ ID NO: 58;
(e) SEQ ID NO: 59;
(f) SEQ ID NO: 60;
(g) SEQ ID NO: 61;
(h) SEQ ID NO:62; and
(i) SEQ ID NO: 63.

7. An antibody or antigen-binding fragment thereof that specifically binds to the spike protein of SARS-CoV-2 comprising a variable heavy chain (VH) and a variable light chain (VL), wherein the variable light chain (VL) amino acid sequence selected from the group consisting of:
(a) SEQ ID NO: 64;
(b) SEQ ID NO: 65;
(c) SEQ ID NO: 66;
(d) SEQ ID NO: 67;
(e) SEQ ID NO: 68;
(f) SEQ ID NO: 69;
(g) SEQ ID NO: 70;
(h) SEQ ID NO: 71; and
(i) SEQ ID NO: 72.

8. The antibody or antigen-binding fragment thereof of any one of claims 1-7, wherein the antibody or antigen-binding fragment thereof comprises the variable heavy chain (VH) amino acid sequence and the variable light chain (VL) amino acid sequence selected from the group consisting of:
(a) SEQ ID NO: 55 and SEQ ID NO: 64, respectively;
(b) SEQ ID NO: 56 and SEQ ID NO: 65, respectively;
(c) SEQ ID NO: 57 and SEQ ID NO: 66, respectively;
(d) SEQ ID NO: 58 and SEQ ID NO: 67, respectively;
(e) SEQ ID NO: 59 and SEQ ID NO: 68, respectively;
(f) SEQ ID NO: 60 and SEQ ID NO: 69, respectively;
(g) SEQ ID NO: 61 and SEQ ID NO: 70, respectively;
(h) SEQ ID NO:62 and SEQ ID NO: 71, respectively; and
(i) SEQ ID NO: 63 and SEQ ID NO: 72, respectively.

9. The antibody or antigen-binding fragment thereof of any one of claims 1-8, wherein the antibody or antigen-binding fragment thereof cross-reacts with SARS-CoV.

10. The antibody or antigen-binding fragment thereof of any one of claims 1-9, wherein the antibody or antigen-binding fragment comprises a heavy chain constant region.

11. The antibody or antigen-binding fragment thereof of claim 10, wherein the heavy chain constant region is selected from the group consisting of human immunoglobulins IgG and IgM.

12. The antibody or antigen-binding fragment thereof of any one of claims 1-11, wherein the antibody or antigen-binding fragment comprises a light chain constant region.

13. The antibody or antigen-binding fragment thereof of claim 12, wherein the light chain constant region is a kappa light chain constant region.

14. The antibody or antigen-binding fragment thereof of claim 12, wherein the light chain constant region is lambda light chain constant region.

15. The antibody or antigen binding fragment thereof of any one of claims 1-14, which is a full length antibody.

16. The antibody or antigen binding fragment thereof of any one of claims 1-14, which is an antigen-binding fragment.

17. The antigen binding fragment of claim 16, wherein the antigen binding fragment comprises a Fab, Fab′, F(ab′)₂, single chain Fv (scFv), disulfide linked Fv, V-NAR domain, IgNar, IgGΔCH2, minibody, F(ab′)₃, tetrabody, triabody, diabody, single-domain antibody, (scFv)₂, or scFv-Fc.

18. The antibody or antigen binding fragment thereof of any one of claims 1-17, wherein the antibody or antigen-binding fragment is isolated.

19. The antibody or antigen binding fragment thereof of any one of claims 1-18, wherein the antibody or antigen-binding fragment is monoclonal.

20. The antibody or antigen binding fragment thereof of any one of claims 1-19, wherein the antibody or antigen-binding fragment is recombinant.

21. The antibody or antigen-binding fragment thereof of any one of claims 1-20, wherein the antibody or antigen-binding fragment does not neutralize SARS-CoV-2.

22. The antibody or antigen-binding fragment thereof of any one of claims 1-20, wherein the antibody or antigen-binding fragments does not neutralize a pseudovirus of SARS-CoV-2.

23. The antibody or antigen-binding fragment thereof of any one of claims 1-22, further comprising a detectable label.

24. The antibody or antigen-binding fragment thereof of claim 23, wherein said label is selected from the group consisting of enzyme label, gold particles, immunofluorescent label, chemiluminescent label, phosphorescent label, radiolabel, avidin/biotin, colored particles and magnetic particles.

25. The antibody or antigen-binding fragment thereof of claim 24, wherein said label is detected by enzyme immunoassay, lateral flow test, radioimmunoassay, Western blot assay, immunofluorescent assay, immunoprecipitation assay, chemiluminescent assay, cytometry, or immunohistochemical assay.

26. A polypeptide comprising the amino acid sequence of SEQ ID NO: 101.

27. The polypeptide of claim 26, wherein the polypeptide is isolated.

28. The polypeptide of claim 26 or 27, wherein the polypeptide is recombinantly produced or chemically synthesized.

29. A trimer comprising the polypeptide of claim 27 or 28, optionally wherein the trimer is isolated.

30. An isolated polynucleotide comprising a nucleic acid molecule encoding the polypeptide of claim 26 or 27.

31. An isolated polynucleotide comprising a nucleic acid molecule encoding the heavy chain variable region and/or a nucleic acid molecule encoding the light chain variable region of the antibody or antigen-binding fragment thereof of any one of claims 1-25.

32. An isolated vector comprising the polynucleotide of claim 30 or 31.

33. A host cell comprising the polynucleotide of claim 30 or 31, the vector of claim 32, or a first vector comprising a nucleic acid molecule encoding the heavy chain variable region and a second vector comprising a nucleic acid molecule encoding the light chain variable region of the antibody or antigen-binding fragment thereof of any one of claims 1-25.

34. A method of making the antibody or antigen-binding fragment thereof of any one of claims 1-25 or the protein of any one of claims 27-27 comprising (a) culturing the cell of claim 33; and (b) isolating the antibody or antigen-binding fragment thereof or the protein from the cultured cell.

35. A method for detecting an anti-SARS-CoV-2 antibody or antigen-binding fragment thereof in a sample comprising contacting the sample with the polypeptide of any one of claims 26-28 or the trimer of claim 29, optionally wherein the method further comprises detecting binding between the antibody or antigen-antigen binding fragment and the polypeptide or the trimer.

36. The method of claim 35, wherein the sample is a biological sample.

37. The method of claim 35 or 36, wherein the detecting method is an enzyme linked immunosorbent assay (ELISA).

38. The method of claim 35 or 36, wherein the detecting method is a lateral flow assay.

39. The method of any one of claims 35-37, wherein the sample is from a human subject.

40. The method of any one of claims 36-39, wherein the sample contains IgG antibodies or antigen-binding fragments thereof.

41. The method of any one of claims 36-40, wherein the sample contains IgM antibodies or antigen-binding fragments thereof.

42. The method of any one of claims 35-41 comprising use of the antibody or antigen-binding fragment thereof of any one of claims 1-25 as a control.

43. A kit comprising (i) the SARS-CoV-2 spike protein of any one of claims 26-28, the trimer of claim 29, or the polynucleotide of claim 30 and (ii) an antibody or antigen-binding fragment thereof that binds to SARS-CoV-2.

44. The kit of claim 43, wherein the antibody or antigen-binding fragment thereof is the antibody or antigen-binding fragment thereof of any one of claims 1-25.

45. A kit comprising the antibody or antigen-binding fragment thereof of any one of claims of any one of claims 1-25 and a SARS-Co-V2 spike protein antigen.

46. The kit of any one of claims 43-45 further comprising a notice that reflects approval for use or sale.
